(19) United States
(12) Patent Application Publication
(10) Pub. No.: US 2025/0299799 A1
(43) Pub. Date: Sep. 25, 2025

(54) DIGITAL THERAPEUTIC METHOD AND SYSTEM EMPLOYING NUTRITIONAL COGNITIVE BEHAVIORAL THERAPY

(71) Applicant: Click Therapeutics, Inc., New York, NY (US)

(72) Inventors: Kevin Appelbaum, Bend, OR (US); Mark Berman, Austin, TX (US); Andres Camacho, Lafayette, CA (US); Kristin Wynholds, Lafayette, CA (US); Anitra Appa, San Francisco, CA (US); Martha Simmons, Belmont, CA (US); Prapti Mehta, San Anselmo, CA (US)

(73) Assignee: Click Therapeutics, Inc., New York, NY (US)

(21) Appl. No.: 19/231,379

(22) Filed: Jun. 6, 2025

Related U.S. Application Data
(63) Continuation of application No. PCT/US2024/019259, filed on Mar. 8, 2024.
(60) Provisional application No. 63/450,954, filed on Mar. 8, 2023.

(51) Int. Cl. G16H 20/60; A61B 5/00; A63B 24/00; G09B 5/02; G16H 20/30

(52) U.S. Cl. CPC G16H 20/60; A61B 5/4839; A61B 5/486; A61B 5/4866; A63B 24/0075; G09B 5/02; G16H 20/30

(57) ABSTRACT

Nutritional Cognitive Behavioral Therapy (Nutritional-CBT) is provided for the treatment of patients with type 2 diabetes and other cardiometabolic diseases, addressing common maladaptive thinking and beliefs pertaining to diet and lifestyle in a digitally-delivered therapy personalized to the individual patient using artificial intelligence (AI)/machine learning (ML) driven feed-back loops. Systems, methods, and computer-readable media described herein can include providing, by one or more processors, a digital therapeutic application including one or more lessons or activities. The one or more processors can collect at least one response or biometric data from the user. The one or more processors can generate, using a machine-learning model, one or more goals for the user to achieve or a progress overview.

### CROSS-REFERENCE TO RELATED APPLICATIONS

The present application is a continuation application filed under 35 U.S.C. § 111(a) of International Patent Application No. PCT/US2024/019259, filed on Mar. 8, 2024, which claims the benefit of U.S. Provisional Application No. 63/450,954, filed on Mar. 8, 2023, both of which are incorporated herein by reference in their entirety and for all purposes.

### BACKGROUND

The above-mentioned patent applications describe a digital therapeutic platform targeting maladaptive beliefs and behavioral factors contributing to type 2 diabetes and related cardiometabolic disorders. These behavioral factors can negatively impact adherence to physical activity regimens, lifestyle modifications, and therapeutic exercises. There remains a need to provide a system that integrates digital therapeutic content with dynamic exercise interventions, personalized body treatment recommendations, and feedback mechanisms that adapt to the user's physical and behavioral status. The present disclosure addresses these challenges by providing systems and methods for improving adherence to therapeutic exercises, personalized movement interventions, and real-time monitoring of body treatment progress.

### SUMMARY

The present disclosure provides several improvements to a digital therapeutic employing Nutritional Cognitive Behavioral Therapy (Nutritional-CBT) to treat patients with type 2 diabetes and other cardiometabolic diseases, e.g. optimizing the timing and content of intervening prompts and notifications to increase patient engagement, and enhancing behavorial priming features to further incentivize and motivate individual patients. In one aspect, the disclosure provides progress reports as part of an algorithmic feedback loop for setting goals for patients. In another aspect, the disclosure determines when patients are not making sufficient progress toward achieving their goals, and optimizes both the content and timing of prompts and notifications to re-engage patients with the digital therapeutic. In yet another aspect, the disclosure determines patients' progress toward achieving their goals, and either repeats a current therapy lesson or an earlier one of the therapy lessons, or even skips one or more of the therapy lessons following the current one.

In one aspect, the disclosure provides a computer-implemented method for dynamically treating a subject having a cardiometabolic disorder, the method comprising: providing, by at least one processor, a digital therapeutic to said subject, the digital therapeutic comprising: a series of therapy lessons, wherein each therapy lesson addresses one or more of maladaptive beliefs relating to dietary and/or lifestyle behaviors of said subject; at least one interactive skill-based exercise for each of said therapy lessons to reinforce an improvement with respect to said one or more maladaptive beliefs and/or to initiate a new dietary and/or lifestyle behavior; the method further comprising: collecting responses from said subject during each said therapy lesson and/or said at least one interactive skill-based exercise, and collecting biometric data from said subject during and/or after each said therapy lesson and/or said at least one interactive skill-based exercise; responsive to the collecting, using at least one treatment processor, providing a progress report characterizing progress by said subject to address said one or more of said maladaptive beliefs, said progress report comprising data relating to the subject's meals and exercise, and data relating to the subject's medication and biometrics; and responsive to the progress report, recommending one or more goals for said subject to achieve between a current and a next therapy lesson in the series of therapy lessons, the recommending comprising inputting one or more pieces of data in the progress report to one or more algorithms, including one or more machine learning algorithms, so as to provide one or more recommendations for the one or more goals, and wherein the one or more machine learning algorithms are trained using responses and biometric data from a plurality of subjects. In embodiments, the one or more goals comprise one or more of diet, exercise and medication.

In embodiments, the method further comprises modifying, for the subject, a subsequent one of said series of therapy lessons and/or said at least one interactive skill-based exercises using said at least one treatment processor, wherein the modifying applies one or more machine learning algorithms trained using responses and biometric data from a plurality of subjects.

In embodiments, the maladaptive belief is selected from the group comprising or consisting of: ability of the subject to change and/or control behaviors; beliefs of the subject regarding nutrients, optionally including macronutrients, and importance of various food types; or beliefs of the subject regarding experiences in eating and/or exercising.

In embodiments, the therapy lesson can be specific to the particular condition that the digital therapeutic is treating (for example, type 2 diabetes or another cardiometabolic disorder), or to understanding, addressing, and/or controlling particular human physiological attributes (for example, blood sugar, blood pressure, heartbeat, weight), or to understanding, addressing, and/or controlling particular human physiological responses (for example, hunger, thirst, craving), or developing certain desirable behaviors (for example, stress reduction, exercise, rest, sleep).

In embodiments, the topic of the therapy lesson is selected from the group comprising or consisting of: exploring beliefs, ideas about health, Type 2 Diabetes, blood sugar, carbohydrates, protein, affordability, exercise, hunger, weight, comfort food, control, loyalty, ability to change, healing, power of beliefs, stress, response to stress, sleep, connection, opportunity, meaning, purpose, strength/resistance exercise, caring for ourselves, empowerment, craving, and/or evolving. In embodiments, the series of therapy lessons comprises two or more, three or more, four or more, five or more, ten or more, fifteen or more, twenty or more, twenty-five or more, or all of the foregoing topics.

In embodiments, the method further comprises generating one or more personalized notifications and communicating the one or more personalized notifications to said subject, the one or more personalized notifications selected from the group consisting of guidance, treatment score, reminders, nudges, and rewards.

In embodiments, the one or more machine learning algorithms is selected from the group comprising or consisting of Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, AdaBoost, or an artificial neural network selected from the group comprising or consisting of convolutional neural networks (CNN), recurrent neural networks (RNN), and recurrent convolutional neural networks (RCNN).

In embodiments, one or more of the series of therapy lessons are interactive, and the subject inputs information into the digital therapeutic either voluntarily or as the digital therapeutic provides a prompt. In embodiments, the information is selected from the group consisting of audio recordings, video recordings, photographs, journal entries, or other written text.

In embodiments, the digital therapeutic resides in a smart device selected from the group consisting of a tablet or a smartphone.

In embodiments, the collecting comprises the subject entering the subject's biometric data. In the present application, “biometric” is not limited to physically identifying information, as in a security context, but includes a wide range of physical measurements which can provide information about a subject's condition, as ordinarily skilled artisans will appreciate. In embodiments, the method further comprises providing biometric notifications in response to entry of the subject's biometric data, optionally wherein the biometric notifications indicate danger levels. In embodiments, the method further comprises determining one or more treatment changes and/or behavioral modifications for the subject.

In embodiments, the disclosure provides a computer system for dynamically adjusting maladaptive beliefs in a subject, the system comprising: a processor for processing a set of instructions; and a non-transitory computer-readable storage medium for storing the set of instructions, wherein the instructions, when executed by the processor, perform a method for dynamically adjusting maladaptive beliefs in a subject comprising any or all of the embodiments of that method as just described.

In embodiments, the disclosure provides a non-transitory computer-readable storage medium for storing a set of instructions, wherein the instructions, when executed by the processor, perform a method for dynamically adjusting maladaptive beliefs in a subject comprising any or all of the embodiments of that method as just described.

In another aspect, the disclosure provides a computer-implemented method for dynamically treating a subject having a cardiometabolic disorder, comprising: providing, by at least one processor, a digital therapeutic to said subject, the digital therapeutic comprising: a series of therapy lessons, wherein each therapy lesson addresses one or more of maladaptive beliefs relating to dietary and/or lifestyle behaviors of said subject; at least one interactive skill-based exercise for each of said therapy lessons to reinforce an improvement with respect to said one or more maladaptive beliefs and/or to initiate a new dietary and/or lifestyle behavior; the method further comprising: collecting responses from said subject during each said therapy lesson and/or said at least one interactive skill-based exercise, and collecting biometric data from said subject during and/or after each said therapy lesson and/or said at least one interactive skill-based exercise; responsive to the collecting, using at least one treatment processor, identifying one or more goals for said subject to achieve between a current and a next therapy lesson in the series of therapy lessons, wherein the identifying applies one or more algorithms, including one or more machine learning algorithms, and wherein the one or more machine learning algorithms are trained using responses and biometric data from a plurality of subjects; responsive to the collecting, using at least one treatment processor, reviewing said subject's progress toward achieving the one or more goals; responsive to a determination that said subject is not making sufficient progress toward achieving the one or more goals, determining when to send the subject one or more personalized notifications to encourage the subject to provide increased effort to complete one of said therapy lessons and/or perform the at least one interactive skill-based exercise; and responsive to the determining, sending the subject the one or more personalized notifications to encourage the subject to provide increased effort to complete said one of said therapy lessons and/or perform the at least one interactive skill-based exercise, wherein the determining and the sending employs the one or more algorithms, including the one or more machine learning algorithms, so as to identify a content, timing and/or frequency of the one or more personalized notifications; wherein the one or more machine learning algorithms are trained using responses and biometric data from a plurality of subjects.

In embodiments, the one or more personalized notifications are selected from the group comprising or consisting of reminders, nudges, and rewards. In embodiments, reminders comprise push notifications to said subject regarding one of said therapy lessons and/or one of said skill-building exercises. In embodiments, nudges comprise notifications to said subject to direct said subject to a correct next therapy lesson and/or skill-building exercise, or to direct said subject to complete and/or initiate an undone therapy lesson and/or skill-building exercise. In embodiments, rewards comprise one or more acknowledgements of successful completion of a therapy lesson and/or skill-building exercise, and/or one or more milestones relating to the subject's meals and/or exercise, and/or relating to the subject's medication and biometrics.

In embodiments, the cardiometabolic disorder is selected from the group comprising or consisting of type 2 diabetes, gestational diabetes, hypertension, obesity, dyslipidemia, hyperlipidemia, hypertriglyceridemia, non-alcoholic fatty liver disease, non-alcoholic steatohepatitis, hypercholesterolemia and familial hypercholesterolemia, heart disease, coronary artery disease, or chronic kidney disease.

In embodiments, the method further comprises dynamically adjusting the goals for the subject between consecutive therapy lessons in said series using the at least one treatment processor, wherein the dynamic adjustment applies one or more machine learning algorithms trained using responses and biometric data from a plurality of subjects. In embodiments, the one or more goals comprise one or more of diet, exercise and medication.

In embodiments, the method further comprises modifying, for the subject, a subsequent one of said series of therapy lessons and/or said at least one interactive skill-based exercises using said at least one treatment processor, wherein the modifying applies one or more machine learning algorithms trained using responses and biometric data from a plurality of subjects.

In embodiments, the maladaptive belief is selected from the group comprising or consisting of: ability of the subject to change and/or control behaviors; beliefs of the subject regarding nutrients, optionally including macronutrients, and importance of various food types; or beliefs of the subject regarding experiences in eating and/or exercising.

In embodiments, the therapy lesson can be specific to the particular condition that the digital therapeutic is treating (for example, type 2 diabetes or another cardiometabolic disorder), or to understanding, addressing, and/or controlling particular human physiological attributes (for example, blood sugar, blood pressure, heartbeat, weight), or to understanding, addressing, and/or controlling particular human physiological responses (for example, hunger, thirst, craving), or developing certain desirable behaviors (for example, stress reduction, exercise, rest, sleep).

In embodiments, the topic of the therapy lesson is selected from the group comprising or consisting of: exploring beliefs, ideas about health, Type 2 Diabetes, blood sugar, carbohydrates, protein, affordability, exercise, hunger, weight, comfort food, control, loyalty, ability to change, healing, power of beliefs, stress, response to stress, sleep, connection, opportunity, meaning, purpose, strength/resistance exercise, caring for ourselves, empowerment, craving, and/or evolving. In embodiments, the series of therapy lessons comprises two or more, three or more, four or more, five or more, ten or more, fifteen or more, twenty or more, twenty-five or more, or all of the foregoing topics.

In embodiments, the one or more machine learning algorithms is selected from the group comprising or consisting of Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, AdaBoost, or an artificial neural network selected from the group comprising or consisting of convolutional neural networks (CNN), recurrent neural networks (RNN), and recurrent convolutional neural networks (RCNN).

In embodiments, one or more of the series of therapy lessons are interactive, and the subject inputs information into the digital therapeutic either voluntarily or as the digital therapeutic provides a prompt. In embodiments, the information is selected from the group consisting of audio recordings, video recordings, photographs, journal entries, or other written text.

In embodiments, the digital therapeutic resides in a smart device selected from the group consisting of a tablet or a smartphone.

In embodiments, the collecting comprises the subject entering the subject's biometric data. In embodiments, the method further comprises providing biometric notifications in response to entry of the subject's biometric data, optionally wherein the biometric notifications indicate danger levels. In embodiments, the method further comprises determining one or more treatment changes and/or behavioral modifications for the subject.

In embodiments, the disclosure provides a computer system for dynamically adjusting a treatment plan for a subject having a cardiometabolic disorder, the system comprising: a processor for processing a set of instructions; and a non-transitory computer-readable storage medium for storing the set of instructions, wherein the instructions, when executed by the processor, perform a method for dynamically adjusting a treatment plan for a subject having a cardiometabolic disorder comprising any or all of the embodiments of that method as just described.

In embodiments, the disclosure provides a non-transitory computer-readable storage medium for storing a set of instructions, wherein the instructions, when executed by the processor, perform a method for dynamically adjusting a treatment plan for a subject having a cardiometabolic disorder comprising any or all of the embodiments of that method as just described.

In another aspect, the disclosure provides a computer-implemented method for dynamically adjusting a treatment plan for a subject having a cardiometabolic disorder, the method comprising: providing, by at least one processor, a digital therapeutic to said subject, the digital therapeutic comprising a treatment plan comprising: a series of therapy lessons, wherein each therapy lesson addresses one or more of maladaptive beliefs relating to dietary and/or lifestyle behaviors of said subject; at least one interactive skill-based exercise for each of said therapy lessons to reinforce an improvement with respect to said one or more maladaptive beliefs and/or to initiate a new dietary and/or lifestyle behavior; the method further comprising: collecting responses from said subject during each said therapy lesson and/or said at least one interactive skill-based exercise, and collecting biometric data from said subject during and/or after each said therapy lesson and/or said at least one interactive skill-based exercise; responsive to the collecting, using at least one treatment processor, identifying one or more goals for said subject to achieve between a current and a next therapy lesson in the series of therapy lessons, wherein the identifying applies one or more algorithms, including one or more machine learning algorithms, and wherein the one or more machine learning algorithms are trained using responses and biometric data from a plurality of subjects; responsive to the collecting, using at least one treatment processor, reviewing said subject's progress toward achieving the one or more goals; and responsive to a determination of said subject's progress toward achieving the one or more goals, performing one of the following to dynamically adjust the treatment plan: repeating the current therapy lesson; repeating an earlier one of the series of therapy lessons; or skipping one or more of the series of therapy lessons following the current therapy lesson; wherein the determination employs the one or more algorithms, including the one or more machine learning algorithms.

In embodiments, the cardiometabolic disorder is selected from the group comprising or consisting of type 2 diabetes, gestational diabetes, hypertension, obesity, dyslipidemia, hyperlipidemia, hypertriglyceridemia, non-alcoholic fatty liver disease, non-alcoholic steatohepatitis, hypercholesterolemia and familial hypercholesterolemia, heart disease, coronary artery disease, or chronic kidney disease.

In embodiments, the method further comprises dynamically adjusting the goals for the subject between consecutive therapy lessons in said series using the at least one treatment processor, wherein the dynamic adjustment applies one or more machine learning algorithms trained using responses and biometric data from a plurality of subjects. In embodiments, the one or more goals comprise one or more of diet, exercise and medication.

In embodiments, the method further comprises modifying, for the subject, a subsequent one of said series of therapy lessons and/or said at least one interactive skill-based exercises using said at least one treatment processor, wherein the modifying applies one or more machine learning algorithms trained using responses and biometric data from a plurality of subjects.

In embodiments, the maladaptive belief is selected from the group comprising or consisting of: ability of the subject to change and/or control behaviors; beliefs of the subject regarding nutrients, optionally including macronutrients, and importance of various food types; or beliefs of the subject regarding experiences in eating and/or exercising.

In embodiments, the therapy lesson can be specific to the particular condition that the digital therapeutic is treating (for example, type 2 diabetes or another cardiometabolic disorder), or to understanding, addressing, and/or controlling particular human physiological attributes (for example, blood sugar, blood pressure, heartbeat, weight), or to understanding, addressing, and/or controlling particular human physiological responses (for example, hunger, thirst, craving), or developing certain desirable behaviors (for example, stress reduction, exercise, rest, sleep).

In embodiments, the topic of the therapy lesson is selected from the group comprising or consisting of: exploring beliefs, ideas about health, Type 2 Diabetes, blood sugar, carbohydrates, protein, affordability, exercise, hunger, weight, comfort food, control, loyalty, ability to change, healing, power of beliefs, stress, response to stress, sleep, connection, opportunity, meaning, purpose, strength/resistance exercise, caring for ourselves, empowerment, craving, and/or evolving. In embodiments, the series of therapy lessons comprises two or more, three or more, four or more, five or more, ten or more, fifteen or more, twenty or more, twenty-five or more, or all of the foregoing topics.

In embodiments, the method further comprises generating one or more personalized notifications and communicating the one or more personalized notifications to said subject, the one or more personalized notifications selected from the group consisting of guidance, progress reporting, treatment score, reminders, nudges, and rewards.

In embodiments, the one or more machine learning algorithms is selected from the group comprising or consisting of Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, AdaBoost, or an artificial neural network selected from the group comprising or consisting of convolutional neural networks (CNN), recurrent neural networks (RNN), and recurrent convolutional neural networks (RCNN).

In embodiments, one or more of the series of therapy lessons are interactive, and the subject inputs information into the digital therapeutic either voluntarily or as the digital therapeutic provides a prompt. In embodiments, the information is selected from the group consisting of audio recordings, video recordings, photographs, journal entries, or other written text.

In embodiments, the digital therapeutic resides in a smart device selected from the group consisting of a tablet or a smartphone.

In embodiments, the collecting comprises the subject entering the subject's biometric data. In embodiments, the method further comprises providing biometric notifications in response to entry of the subject's biometric data, optionally wherein the biometric notifications indicate danger levels. In embodiments, the method further comprises determining one or more treatment changes and/or behavioral modifications for the subject.

In embodiments, the disclosure provides a computer system for dynamically treating a subject having a cardiometabolic disorder, the system comprising: a processor for processing a set of instructions; and a non-transitory computer-readable storage medium for storing the set of instructions, wherein the instructions, when executed by the processor, perform a method for dynamically treating a subject having a cardiometabolic disorder comprising any or all of the embodiments of that method as just described.

In embodiments, the disclosure provides a non-transitory computer-readable storage medium for storing a set of instructions, wherein the instructions, when executed by the processor, perform a method for dynamically treating a subject having a cardiometabolic disorder comprising any or all of the embodiments of that method as just described.

Also provides is a computer-implemented method for dynamically treating a subject having a cardiometabolic disorder, the method comprising: providing, by at least one processor, a digital therapeutic to said subject, the digital therapeutic comprising: a series of therapy lessons, wherein each therapy lesson addresses one or more of maladaptive beliefs relating to dietary and/or lifestyle behaviors of said subject; at least one interactive skill-based exercise for each of said therapy lessons to reinforce an improvement with respect to said one or more maladaptive beliefs and/or to initiate a new dietary and/or lifestyle behavior; the method further comprising: collecting responses from said subject during each said therapy lesson and/or said at least one interactive skill-based exercise, and collecting biometric data from said subject during and/or after each said therapy lesson and/or said at least one interactive skill-based exercise; responsive to the collecting, using at least one treatment processor, identifying one or more goals for said subject to achieve between a current and a next therapy lesson in the series of therapy lessons, wherein the identifying applies one or more algorithms, including one or more machine learning algorithms, and wherein the one or more machine learning algorithms are trained using responses and biometric data from a plurality of subjects; responsive to the collecting, using at least one treatment processor, reviewing said subject's progress toward achieving the one or more goals; and responsive to a determination of said subject's progress toward achieving the one or more goals, performing one of the following to dynamically treat the subject: repeating the current therapy lesson; repeating an earlier one of the series of therapy lessons; or skipping one or more of the series of therapy lessons following the current therapy lesson; wherein the determination employs the one or more algorithms, including the one or more machine learning algorithms.

In embodiments, the disclosure provides a computer system for dynamically treating a subject having a cardiometabolic disorder, the system comprising: a processor for processing a set of instructions; and a non-transitory computer-readable storage medium for storing the set of instructions, wherein the instructions, when executed by the processor, perform a method for dynamically treating a subject having a cardiometabolic disorder comprising any or all of the embodiments of that method as just described.

In embodiments, the disclosure provides a non-transitory computer-readable storage medium for storing a set of instructions, wherein the instructions, when executed by the processor, perform a method for dynamically treating a subject having a cardiometabolic disorder comprising any or all of the embodiments of that method as just described.

### BRIEF DESCRIPTION OF THE DRAWINGS

Aspects of the present disclosure now will be described in detail with reference to the accompanying drawings, in which:

FIG. 1A is a diagram providing an overview of aspects of embodiments, and FIGS. 1B-1D are flowcharts for aspects of FIG. 1A;

FIG. 2 is a bar chart showing results of varying degrees of use of nutritional CBT and corresponding FBG results;

FIG. 3 is a bar chart showing results of varying degrees of presence of plant-based nutrition in patient diets, and corresponding FBG results;

FIG. 4 is a bar chart showing results of varying degrees of exercise and corresponding FBG results;

FIG. 5 is a bar chart showing results of varying degrees of use of nutritional CBT and corresponding weight loss results;

FIG. 6 is a bar chart showing patient feedback regarding a digital therapeutic applying nutritional CBT without benefit of AI/ML;

FIG. 7 is a high-level block diagram depicting aspects of a nutritional CBT system according to an embodiment;

FIG. 8 is a high-level block diagram of one of the elements of a nutritional CBT system according to an embodiment.

FIG. 9 is a graph showing a greater reduction in A1c for patients on a nutritional CBT system as compared to control group;

FIG. 10 is a bar chart showing gradual and steady improvements in fasting blood glucose levels, for patients on a nutritional CBT system as disclosed herein;

FIG. 11 is a graph showing trends in fasting blood glucose level for three different therapies (a nCBT system as herein disclosed, GLP1, and SGLT2);

FIG. 12 is a bar chart showing that Cardiovascular Outcome Trials (CVOTs) show lower relative A1c reduction compared with new drug pivotal for same drug;

FIG. 13 is a bar chart showing that higher dose of nCBT lessons completed is associated with larger A1c improvements at 180 days;

FIG. 14 is a graph showing that a higher nCBT dose subgroup shows substantially greater A1c improvement compared to standard of care (SOC) control group;

FIG. 15 is a bar chart illustrating significant improvements in A1c levels in patients on a nCBT system as described herein, despite use of fewer diabetes medications;

FIG. 16 is bar chart showing that patients on a nCBT system as herein disclosed show a range of large improvements in A1c levels at 180 days;

FIG. 17 is a bar chart showing that higher nCBT dose as herein described is associated with larger improvements, but not higher rates of adverse events (AEs);

FIG. 18 is a bar chart illustrating that antihyperglycemic medication utilization and healthcare utilization increased more in the SOC control group patients than those on an nCBT system as herein disclosed, over 6 months;

FIG. 19 is a bar chart showing the percent change from baseline in liver fat by MRI-PDFF in participants with an elevated baseline, in which a waterfall plot shows a change from baseline in MRI-PDFF for participants with a baseline PDFF greater than or equal to 10% (n=14). A mean change of −16.2% (P=0.011) was observed using a one-sample Wilcoxon rank sum test;

FIG. 20 is a bar chart showing the change from baseline in alanine transaminase (ALT) in ITT population, in which a waterfall plot shows a change from baseline in ALT for all participants in the ITT population (n=17). A mean change of −17.1 IU/L (P=0.002) was observed in the ITT population using a one-sample t-test. In those with an elevated ATL at baseline (n=13), a mean change of −22.5 IU/L (P=0.001) was observed using a one-sample t-test;

FIG. 21 is a bar chart showing the change in Fast score risk category, with a comparison of the number of participants in the ITT population in each Fast score risk category at baseline and postintervention.

### DETAILED DESCRIPTION

Physicians and other clinicians, as well as scientists and other scientifically-trained professionals, often share data or results of their work, in the form of journal articles, conference reports, and the like. Such dissemination is intended to advance scientific and/or medical knowledge and learning, and enable application of that imparted information to further patient treatment, for example.

There are limits to the extent and amount of information dissemination and consequent improvement in clinical results, however. The practicality of individual physicians and other clinicians, whether in a large teaching hospital or in smaller, more remote locations, to actually take advantage of the disseminated information is limited because there is only so much information that an individual doctor or clinician, or even an assembled team of doctors and/or clinicians, can assimilate and apply, or in the case of individual physicians, even obtain. In addition, different sources may provide supplements or other augments to existing information, requiring individuals or even teams to engage in frequent “refreshing” of knowledge and consequent learning of effects on treatment regiments.

Aspects of the present invention provide practical application to computer technology, to expedite provision of patient treatment, and to improve patient outcomes, in ways that prior sharing of information cannot. The content and delivery mechanisms of nutritional-CBT in accordance with aspects of the present invention leverage experience and data from clinician-patient and health coach-patient interactions among substantial patient populations to distill common maladaptive thinking and beliefs pertaining to diet and lifestyle.

In an embodiment, a digitally-delivered therapy can be widely disseminated to large patient populations, yet personalized to the individual patient using artificial intelligence (AI)/machine learning (ML) driven feedback loops. The subject treatment plans provide patient lessons, skill exercises and goals based on a wide range of data from substantial numbers of patients, reflecting many different combinations of physiological, biometric, and psychological characteristics of those patients and corresponding treatment results, yield far more informed and effective treatments because individual physicians or clinicians, even in large hospitals, are unable to assimilate the data the way an AI/ML system can.

According to aspects of the present invention, digitally-delivered nutritional-CBT involves, among other things, one or more of the following:

Identifying and measuring maladaptive thoughts based on misinformed or false underlying core beliefs (e.g., those related to macronutrient fears, the hedonic nature of eating, physical exertion, other perceived barriers to changing lifestyle) that lead to disease-promoting behaviors;

Replacing these maladaptive core beliefs and thought patterns with adaptive ways of thinking developed from rational reflection;

Providing collaborative (between patient and digital therapeutic) construction of behavioral exercises to test core beliefs and set goals for improvement;

Using additional validated behavioral techniques to enhance a patient's capacity to solve problems, plan behaviors, and cope with interfering emotions or thoughts.

A digital therapeutic according to an embodiment delivers treatment to patients with type 2 diabetes to target behaviors related to achieving glycemic control so as to reduce HbA1c. In an embodiment, the digital therapeutic may be downloaded to a patient's smartphone to deliver nutritional-CBT.

Throughout the description of embodiments of the present invention, terms such as “participant,” “patient,” and “subject” may appear. These terms are used interchangeably throughout.

In an embodiment, the digital therapeutic may ask patients to answer behavioral intake questions, as a behavioral assessment. Questions may focus on current and recent biometric data, eating and drinking habits, exercise habits, and the like. Things like family history, and current family, living, and work situations also may be relevant. Additionally or alternatively, some of the topics in the behavioral intake questions may be pursued in more detail in one or more of the therapy lessons, and/or may be a focus of one or more skill-based exercises that may be associated with the various therapy lessons.

During the behavioral assessment, and/or subsequently during treatment, patients may be asked to assess the presence and strength of their beliefs and perceived barriers to achieving diet and exercise patterns that are sufficient to improve glycemic control. A goal of this behavioral assessment is to help patients identify their unconscious beliefs that may be responsible for poor behavioral habits or may represent barriers to adopting new helpful habits that influence glycemic control. Different patients will respond differently to questions asked. Variations can depend on numerous patient characteristics, including but not limited to age, height, weight, health symptoms, living and eating habits, geography, culture, race, and other demographic, physiological, and psychological characteristics. Aspects of the invention use patient responses to tailor the treatment presented, taking advantage of AI/ML learning from vast quantities of other patient data and responses by analyzing and interpreting data in a way that individual physicians and clinicians are incapable of doing.

AI and ML help to reveal the right treatment pace, intensity and support needed to maximize efficacy for each individual. Examples of AI/ML techniques and algorithms that usefully may be employed include, but are not limited to, Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, and AdaBoost. Ordinarily skilled artisans also will appreciate that certain types of machine learning algorithms, such as convolutional neural networks (CNN) and recurrent neural networks (RNN) (sometimes referred to as long short term memory (LSTM), which employ backpropagation, as well as recurrent convolutional neural networks (RCNN) and other types of artificial neural networks, may be particularly suitable.

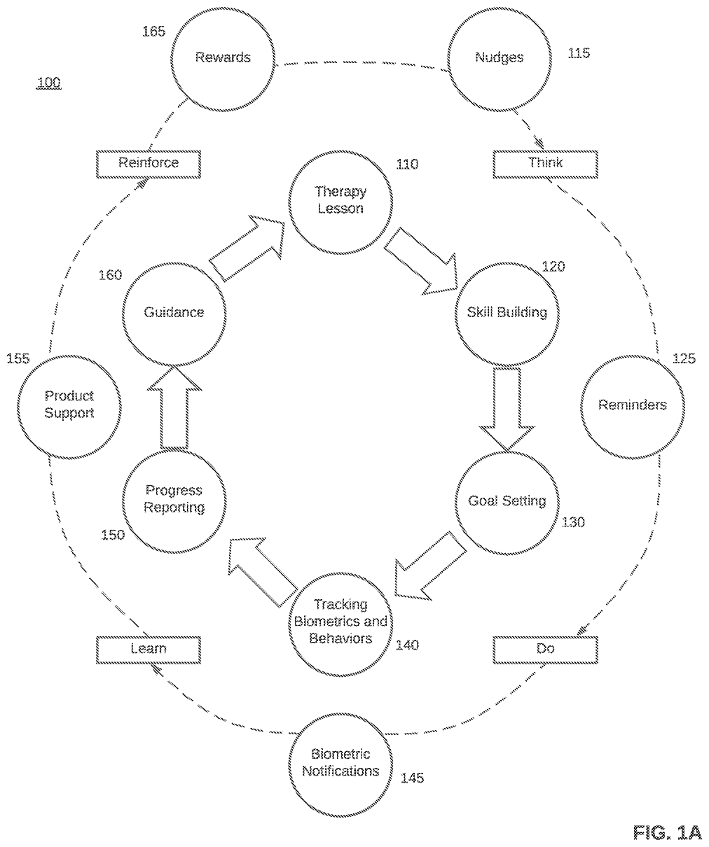

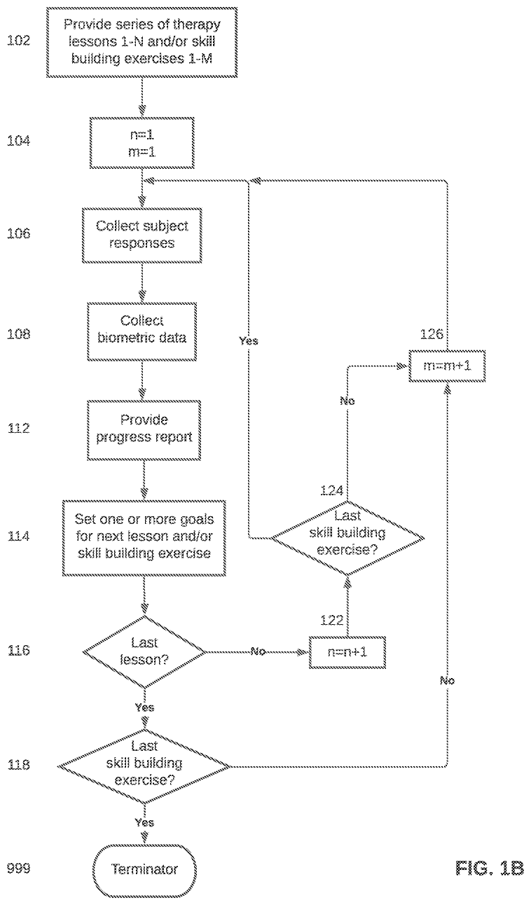

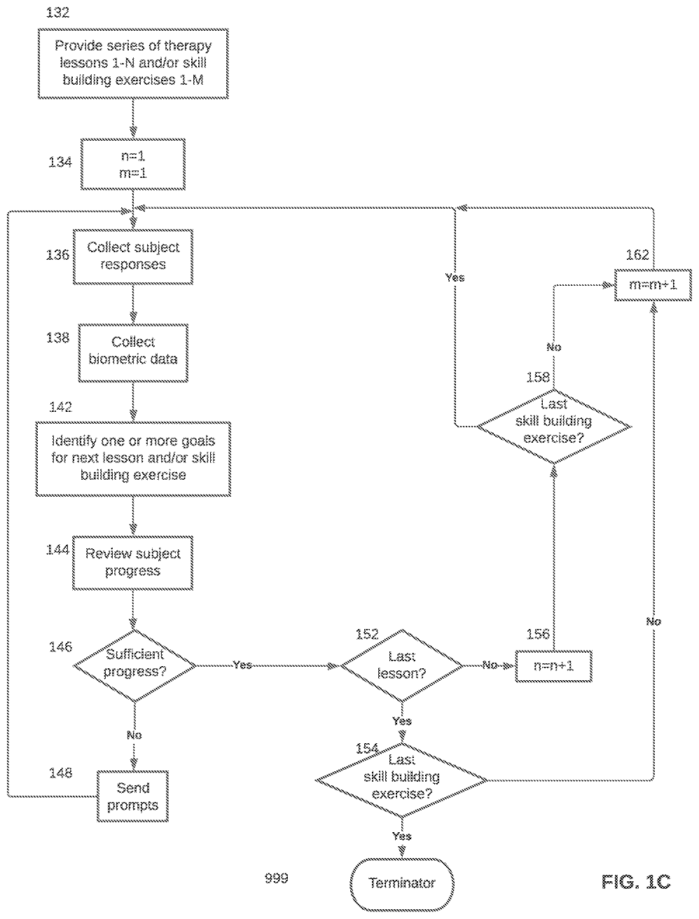

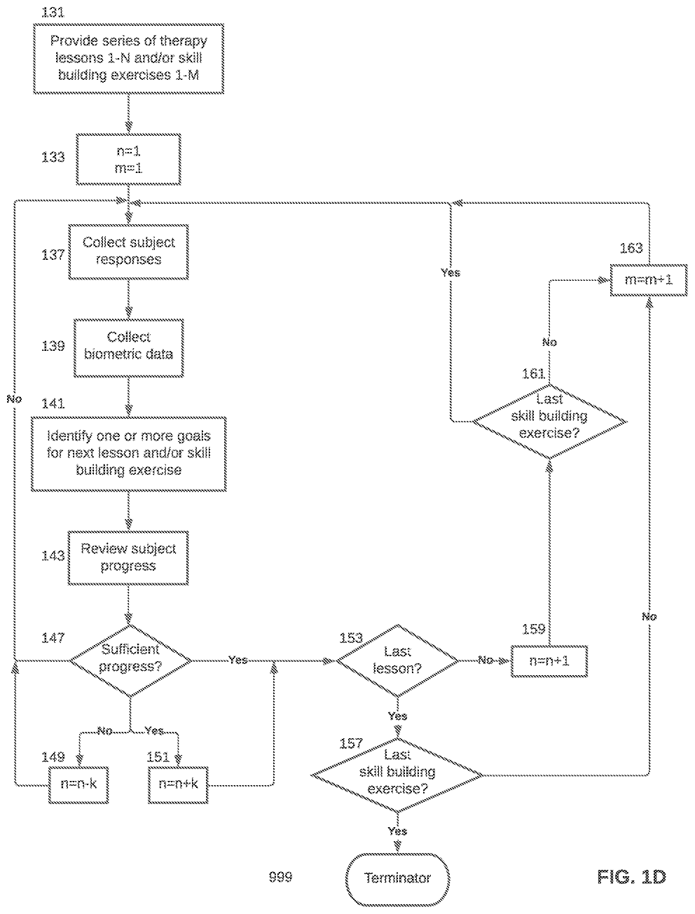

FIG. 1A is a high level diagram providing an overview of what various aspects of embodiments of the invention accomplish, showing two circular diagrams which, in an embodiment, pertain to a therapy lesson cycle 100. The inner circular diagram appears in a somewhat circular form, with a “therapy lesson” bubble at the top. A given therapy lesson can begin a cycle of therapy and subject activity, leading to the next therapy lesson. The inner circular diagram directly involves a therapy lesson 110 which may be part of a series of therapy lessons. The outer circular diagram indirectly involves the various items in the inner circular diagrams, and in several ways provides feedback to a patient or subject. The various elements in the outer circular diagram appear in particular locations relative to elements in the inner circular diagram in FIG. 1A, but it should be noted that the relative locations of the elements in the inner and outer circular diagrams may vary. Various ones of the elements summarized below also will be discussed in more detail herein.

Each therapy lesson 110 can help to isolate and to shift a specific set of beliefs that are barriers to change. Each lesson 110 is or can be interactive. CBT techniques help to identify and shift false beliefs and ideas in a non-threatening, non-judgmental manner. The subject gradually advances from early concepts in early therapy lessons, to allow time for cognitive restructuring before addressing more deeply held beliefs. The therapy creates emotional resilience needed to make enduring changes.

As part of the therapy lesson, the digital therapeutic may provide one or more personalized notifications including, e.g., guidance, progress reporting, treatment scores, reminders, nudges, and rewards. These can, among other things, prompt subjects to think more closely about the lesson; direct subjects to expand their repertoire of activities, perhaps discovering previously undiscovered skills; and even encourage subjects who already are on track to exceed their goals, and/or set higher goals.

These personalized notifications can also give encouragement and suggest activities to reinforce behaviors, as well as help subjects celebrate progress and provide guidance as to what to do next to practice healthy behaviors. In some embodiments, reinforcement and/or celebration is provided when a goal is achieved. In other embodiments, guidance and/or additional actions to take are provided when goals have not been achieved.

Each therapy lesson is followed by a skill-building exercise or module 120. Skill-building modules can reinforce ideas introduced in therapy lessons and put them into practice, initiating new behaviors.

In conjunction with therapy lesson 110, skill-building exercise 120, or other aspects in the inner circular diagram, a subject may receive nudges 115 to prompt them to finish the therapy lesson 110 or to move onto or to finish the skill-building exercise 120.

In an embodiment, in and among the therapy lessons or skill-building modules, or other aspects in the inner circular diagram, subjects may receive reminders about desired activity and/or meal planning. Reminders 125 also can prompt subjects to check in and report biometrics and behaviors, promoting interaction with the digital therapeutic.

In one aspect, reminders and/or nudges may serve to help reestablish a subject's momentum in stepping through therapy lessons and/or skill-building exercises, and/or augment the subject's momentum in stepping through the therapy lessons and/or skill-building exercises. In one aspect, the reminders and/or nudges may comprise one or more visuals to direct the subject to a correct next action (e.g. therapy lesson or skill-building exercise), and/or to steer the subject toward an as-yet incomplete action (e.g. a therapy lesson and/or skill-building exercise). In one aspect, the reason(s) underlying the reminders and/or nudges (e.g. lack of subject interaction with the digital therapeutic) may be used to formulate recommendations of one or more goals for the subject.

As a subject goes through a therapy lesson and works on skill-building, in an embodiment algorithmically prescribed weekly goals as part of goal setting 130 personalize therapy and encourage biometric self-monitoring. In an embodiment, a treatment algorithm may dynamically adjust goals to maximize treatment response, and may provide various personalized feedback loops that help sustain engagement. The subject is able to reflect on accomplishments and activities of the previous week, and review and commit to prescribed goals. In an embodiment, the subject may receive the algorithmically prescribed goals, and alter them, or pick different goals entirely. Interacting with the digital therapeutic can provide guidance to the subject to select achievable goals that are sufficiently ambitious to enable continued progress, but not so ambitious as to be discouraging.

Regular self-monitoring of behaviors and biometrics through tracking 140 can enhance self-efficacy and safety. Providing data to the digital therapeutic in the form of biometric data and behavioral data (in some cases, akin to progress reporting, mentioned below) enables the digital therapeutic to react appropriately to help the subject to continue to progress. In an embodiment, algorithmically calculated biometric notifications or alerts 145 may be triggered when dangerous values or patterns are detected. For example, FBG or blood pressure may be too high or too low. Non-dismissible information from the digital therapeutic can provide more context around the subject's situation and also can provide next steps to follow.

To help subjects remain on track, progress reporting 150 can connect changes in behavior to measurable health improvements week over week. In an embodiment, progress reporting 150 can include a treatment. Progress reporting 150 can show how a subject is doing in relation to each of the biometrics and behaviors they are tracking. Subjects are able to learn about their progress, and about how to make better progress, from the progress reports and treatment score. In embodiments, contents of progress reporting 150 may be used to adjust a subject's treatment plan, adjust a subject's goals, and/or provide nudges 115 or reminders 125 as a result of a progress report. In embodiments, adjusting a subject's treatment plan may entail having the subject repeat one or more therapy lessons 110 or skill-building exercises 120, or skip one or more therapy lessons 110 or skill-building exercises 120, depending on the subject's progress

In an embodiment, subjects may contact Product Support 155 from within the digital therapeutic to help them resolve technical or usability issues with the digital therapeutic, and keep the subjects interested and engaged, avoiding discouragement and/or frustration.

In an embodiment, the digital therapeutic can provide guidance 160 to address common behavioral barriers and misperceptions. When users are off-track or stuck in their progress, guidance 160 can provide additional insights and information. Interactive and visual content can deliver information and insights in a digestible and actionable way.

In embodiments, a subject can receive reinforcement and rewards 165 for various accomplishments. For example, the subject may have consecutive streaks of exercise days or selection of plant-based meals, or may have achieved a drop in FBG or blood pressure or weight. The digital therapeutic can provide rewards to the subject, which in an embodiment may be displayed as trophies in a virtual trophy case. Subjects can review their treatment journey, and can access content, such as journals, voice recordings, and photographs, that they have created during treatment. The rewards and the trophy case display can reinforce and encourage subjects, as well as promote repetition of skills for mastery.

The cycle that FIG. 1 depicts can be repeated as a subject progresses through each lesson. In an embodiment, AI/ML algorithms may determine that a lesson needs to be repeated (if a subject has not shown enough progress during the time between lessons), and/or that too much time has passed since the subject completed a particular therapy lesson and/or skill-building exercise) or that a lesson may be skipped (if a subject has shown enough progress, and/or mastery of a skill to be worked on in the course of a particular lesson), or that lessons should be reordered to optimize the subject's progress. In an embodiment, part of this determination may be based on the provision of one or more reminders and/or nudges.

The subject can experience all of these steps as activities that stimulate conscious thought about what drives their behaviors. Subjects can be introduced to new ideas, and assisted in putting those new ideas into practice. Subject can learn from experience, making them receptive to guidance on how to make behavioral changes. What results is a dynamic adjustment of a subject's maladaptive beliefs.

In an embodiment, the digital therapeutic helps patients understand the steps they should prioritize by presenting them with a treatment plan that summarizes their daily and weekly goals. Each week, the digital therapeutic may ask patients to complete a new nutritional-CBT module, along with one or more skill-based exercises that are related to that particular week's nutritional-CBT module. All patients potentially have access to the same course of nutritional-CBT modules, but as noted above, in an embodiment the course is tailored for patients based on volumes of other patient data and responses. In an embodiment, the nutritional-CBT modules may take between 10-20 minutes to complete. As noted earlier, nutritional-CBT modules may be referred to as “therapy lessons” within the digital therapeutic.

FIG. 1B shows a portion of an overall treatment regimen according to an embodiment. At 102, a subject may be provided with a series of therapy lessons and/or skill-building exercises. At 104, a counter may be initialized to start the series of therapy lessons and/or skill-building exercises. Depending on the embodiment, the number of skill-building exercises may be the same as, greater than, or less than the number of therapy lessons.

At 106, the system may collect subject responses of the types discussed herein, associated with one or both of the current therapy lesson and/or current skill-building exercise. At 108, the system may collect biometric data of the types discussed herein. In an embodiment, the subject may enter their own biometric data. At 112, the subject may receive a progress report. At 114, one or more goals may be set for the next therapy lesson and/or skill-building exercise. In an embodiment, the one or more goals may be set algorithmically, as discussed herein, using one or more machine learning algorithms trained using responses and biometric data of other subjects.

At 116, a check is made to determine whether the therapy lesson is the last one in the series of therapy lessons. If so, then at 118 a check is made to determine whether the skill-building exercise is the last one in the series of skill-building exercises. If not, then at 122 the counter for the therapy lessons is incremented.

If at 118, it is determined that the skill-building exercise is the last one, then it is determined that the subject has completed the nCBT regimen, and flow ends. If it is not, then at 126 the counter for the skill-building exercises may be incremented. Flow then returns to 106 for the next therapy lesson and/or skill-building exercise. If at 124 it is determined that the skill-building exercise is the last one, then flow may return to 106 for the next therapy lesson. If it is not the last one, then at 126 the counter for the skill-building exercises may be incremented. Flow then returns to 106 for the next therapy lesson and/or skill-building exercise.

It should be noted that, in embodiments, 116 and 118 may be reversed, as the order is not critical. The remaining elements 122, 124, and 126 may be adjusted accordingly.

FIG. 1C shows a portion of an overall treatment regimen according to an embodiment. At 132, a subject may be provided with a series of therapy lessons and/or skill-building exercises. At 134, a counter may be initialized to start the series of therapy lessons and/or skill-building exercises. Depending on the embodiment, the number of skill-building exercises may be the same as, greater than, or less than the number of therapy lessons.

At 136, the system may collect subject responses of the types discussed herein, associated with one or both of the current therapy lesson and/or current skill-building exercise. At 138, the system may collect biometric data of the types discussed herein. In an embodiment, the subject may enter their own biometric data. At 142, the system may identify one or more goals for the next therapy lesson and/or skill-building exercise. In an embodiment, this identification may be carried out using one or more machine learning algorithms which may be trained with responses and biometric data from other subjects.

At 144, a subject's progress may be reviewed, either during or after a therapy lesson and/or skill-building exercise. If sufficient progress is not being made, then at 148 prompts in the form of personalized notifications may be sent to the subject to encourage the subject to complete the current therapy lesson and/or skill-building exercise. The subject's renewed or further attempt to complete the current therapy lesson and/or skill-building exercise will result in subject's further engagement with the digital therapeutic. From clinical trials which have been performed, and which are discussed herein, it may be understood that there is a good to very good likelihood that increased engagement with the digital therapeutic will result in a better treatment outcome for the subject.

If at 146 it is determined that the subject is making sufficient progress, then at 152, a check is made to determine whether the therapy lesson is the last one in the series of therapy lessons. If so, then at 154 a check is made to determine whether the skill-building exercise is the last one in the series of skill-building exercises. If not, then at 156 the counter for the therapy lessons is incremented.

If at 154, it is determined that the skill-building exercise is the last one, then it is determined that the subject has completed the nCBT regimen, and flow ends. If it is not, then at 162 the counter for the skill-building exercises may be incremented. Flow then returns to 136 for the next therapy lesson and/or skill-building exercise. If at 158 it is determined that the skill-building exercise is the last one, then flow may return to 136 for the next therapy lesson. If it is not the last one, then at 162 the counter for the skill-building exercises may be incremented. Flow then returns to 136 for the next therapy lesson and/or skill-building exercise.

It should be noted that, in embodiments, 152 and 154 may be reversed, as the order is not critical. The remaining elements 156, 158, and 162 may be adjusted accordingly.

FIG. 1D shows a portion of an overall treatment regimen according to an embodiment. At 131, a subject may be provided with a series of therapy lessons and/or skill-building exercises. At 132, a counter may be initialized to start the series of therapy lessons and/or skill-building exercises. Depending on the embodiment, the number of skill-building exercises may be the same as, greater than, or less than the number of therapy lessons.

At 137, the system may collect subject responses of the types discussed herein, associated with one or both of the current therapy lesson and/or current skill-building exercise. At 139, the system may collect biometric data of the types discussed herein. In an embodiment, the subject may enter their own biometric data. At 141, the system may identify one or more goals for the next therapy lesson and/or skill-building exercise. In an embodiment, this identification may be carried out using one or more machine learning algorithms which may be trained with responses and biometric data from other subjects.

At 143, a subject's progress may be reviewed, either during or after a therapy lesson and/or skill-building exercise. If sufficient progress is not being made, then at 149 the counter for therapy lessons may be decremented by one or more, and flow then may return to 137 for a repeat of the therapy lesson and, where applicable, the skill-building exercise. The subject's renewed or further attempt to complete the current therapy lesson and/or skill-building exercise will result in subject's further engagement with the digital therapeutic. From clinical trials which have been performed, and which are discussed herein, it may be understood that there is a good to very good likelihood that increased engagement with the digital therapeutic will result in a better treatment outcome for the subject.

If at 147 it is determined that the subject is making sufficient progress, then at 153, a check is made to determine whether the therapy lesson is the last one in the series of therapy lessons. If so, then at 157 a check is made to determine whether the skill-building exercise is the last one in the series of skill-building exercises. If not, then at 159 the counter for the therapy lessons is incremented.

If at 157, it is determined that the skill-building exercise is the last one, then it is determined that the subject has completed the nCBT regimen, and flow ends. If it is not, then at 163 the counter for the skill-building exercises may be incremented. Flow then returns to 137 for the next therapy lesson and/or skill-building exercise. If at 161 it is determined that the skill-building exercise is the last one, then flow may return to 137 for the next therapy lesson. If it is not the last one, then at 163 the counter for the skill-building exercises may be incremented. Flow then returns to 137 for the next therapy lesson and/or skill-building exercise.

It should be noted that, in embodiments, 153 and 157 may be reversed, as the order is not critical. The remaining elements 159, 161, and 163 may be adjusted accordingly.

In an embodiment, each therapy lesson may address core patient beliefs in one or more of the following areas:
Personal beliefs and barriers, such as those related to a patient's ability to change and control his or her behaviors;
Beliefs about macronutrients and the importance of various food types; Hedonic-related beliefs about pleasant or unpleasant sensations experienced by eating or exercising;
Beliefs about exercise;
Beliefs about sleep, stress and social interaction.

One purpose of these therapy lessons is to bring forth unconscious beliefs, automatic thoughts (the spontaneously occurring verbal or imaginary mental activity that occurs involuntarily in response to a situation), emotions, and attentional bias (the inappropriate focus of attention on specific information over others that distorts perception) into patients' conscious awareness. Additionally, the therapy lessons and associated skill-based exercises can empower patients to challenge these now conscious thoughts and beliefs by recommending more helpful ideas and behavioral practices. These lessons and exercises allow patients to begin correcting false beliefs and replacing them with consciously adopted thoughts and beliefs that drive disease-reversing behaviors.

In an embodiment, each therapy lesson may comprise one or more of the following core steps:
Identifying false or unhelpful beliefs;
Selecting alternative, more helpful beliefs;
Planning one or more related skills to practice.

The first step helps patients to recognize specific false beliefs and to illustrate how these beliefs may promote behaviors that can worsen type 2 diabetes or other cardiometabolic disorders. To help the patient identify and assess specific false beliefs, in an embodiment the system may present, via the digital therapeutic: multiple-choice quiz questions; patient vignettes; and/or contrasting views of false and alternative beliefs.

The second step helps to expose patients to adaptive alternatives to supersede unhelpful thoughts and beliefs. In an embodiment, the system may present a patient with one alternative belief statement at-a-time and may ask the patient to rate how strongly they currently believe the statement to be true for them. The system then may instruct the patient to vocalize the belief statement with which they identify most strongly, and to record (for example, in a video, audio, and/or text-based journal) about a relevant experience. This rating and journaling process can help to reinforce the idea that beliefs and thoughts can be changed, and that consciously choosing to repeat a new adaptive thought or idea is a key step in the process.

The third step helps patients to plan one or more related skills to practice, so as further to reinforce the alternative thoughts learned in a particular therapy lesson. In an embodiment, the system may ask patients to browse up to five skill options and to select one or more options to practice during the week.

As with face-to-face therapy, the patient is prompted to engage with each of these three steps by considering their unique beliefs, ideas, and life experiences. Each lesson also offers a small number of options for completing each step to allow the patient to further personalize the therapeutic process. Patients can also repeat lessons and skills as needed and gain access to future lessons via a lesson library. In an embodiment, the digital therapeutic analyzes patient-generated data from use of the digital therapeutic to automatically provide feedback to the patient about their degree of engagement in the nutritional-CBT process. In an embodiment, the feedback is analyzed in the context of vast quantities of other patient responses about degree of engagement, taking into account patient personal, cultural, and/or health characteristics.

In an embodiment, also as noted earlier, skill exercises may be provided to improve patients' dietary, exercise, or supportive behavioral patterns. Patients' practice of these skills can enhance executive function tasks such as planning, problem-solving, and goal setting. In an embodiment, each therapy lesson explains the rationale and benefits of the skill exercise or exercises in the lesson, in the context of the core belief topic being explored. In an embodiment, lesson topics may include some or all of the following:

TABLE 1

| Lesson | Title |
|---|---|
| 0 | Exploring our Beliefs: Explore the concept that false beliefs can underlie |
| | unhealthy behavior |
| 1 | Ideas about Health/Type 2 Diabetes: Examine ideas that prevent people from |
| | using lifestyle change to improve their health, including type 2 diabetes or other |
| | cardiometabolic diseases |
| 2 | Blood Sugar/Carbohydrates: Examine ideas about carbohydrates and blood sugar |
| | that prevent people with diabetes from eating healthful whole plant foods |
| 3 | Protein: Examine ideas about protein which prevent people from replacing meat, |
| | dairy, and eggs with whole plant foods |
| 4 | Affordability: Address the misconception that eating whole plant foods must be |
| | expensive |
| 5 | Exercise: Address false ideas about exercise which prevent people from |
| | increasing their weekly exercise minutes |
| 6 | Hunger: Address false beliefs about what hunger indicates |
| 7 | Weight: Explore ideas about the impact of moderate weight loss on type 2 |
| | diabetes |
| 8 | Comfort Food: Explore ideas about the emotions that can lead to eating unhealthy |
| | comfort foods |
| 9 | Control: Explore the ideas about the relative values of skillpower and willpower |
| 10 | Loyalty: Address unhelpful ideas about traditions that can prevent successful |
| | lifestyle changes during social functions |
| 11 | Our Ability to Change: Address ideas about mindset and individual ability to |
| | change behavior |
| 12 | Healing: Explore ideas about setbacks during successful life changes |
| 13 | Power of Beliefs: Explore the power of ideas and how mindsets shift during the |
| | program |
| 14 | Stress: Explore the idea that everybody experiences the range of different kinds |
| | of stress |
| 15 | Response to Stress: Explores ideas about developing skills and curating resources |
| | to respond to stress in healthy ways |
| 16 | Sleep: Addresses ideas about restorative sleep and its importance for health |
| 17 | Connection: Explore ideas about authentic connections to others as a key |
| | component of good health |
| 18 | Opportunity: Explore ideas about craving resulting from past experiences rather |
| | than physiologic need |
| 19 | Meaning: Explore the idea that people can create opportunity for themselves even |
| | under difficult circumstances |
| 20 | Purpose: Explore ideas about the possibility of meaning in everything, even in |
| | difficult situations |
| 21 | Strength/Resistance Exercise: Address ideas about having reasons and purpose |
| | for changes beyond personal health |
| 22 | Caring for Ourselves: Explore ideas around building muscle strength and its |
| | importance in health |
| 23 | Empowerment: Explore the idea that caring for ourselves is fundamentally an |
| | attitude, rather than specific actions |
| 24 | Craving: Address the idea of empowerment in terms of transition from external |
| | to internal motivation |
| 25 | Our Evolving Selves: Explore ideas around expected shifts in self-identity that |
| | come with personal growth |

As ordinarily skilled artisans will appreciate from the foregoing list, some of the lessons may be specific to the particular condition that the digital therapeutic is treating (for example, type 2 diabetes or another cardiometabolic disorder), while others may be more broadly applicable to cardiometabolic disorders in general. For example, some of the lessons may be specific to understanding, addressing, and/or controlling particular human physiological attributes (e.g., blood sugar, blood pressure, heartbeat, weight). Some of the lessons may be specific to understanding, addressing, and/or controlling particular human physiological responses (e.g., hunger, thirst, craving). Some of the lessons may be specific to developing certain desirable behaviors (e.g., stress reduction, exercise, rest, sleep).

Remarkably then, the digital therapeutic of the present invention is readily adaptable to the treatment of a broad set of cardiometabolic diseases beyond type 2 diabetes, including gestational diabetes, hypertension, obesity, dyslipidemia, hyperlipidemia, hypertriglyceridemia, non-alcoholic fatty liver disease, non-alcoholic steatohepatitis, hypercholesterolemia and familial hypercholesterolemia, heart disease, coronary artery disease, and chronic kidney disease, and with only minimal changes to most of the lesson plans and/or skill exercises. The subject digital therapeutic thereby leverages and scales the power of targeting and changing core maladaptive beliefs and behaviors underlying a wide range of cardiometabolic diseases.

In an embodiment, a patient may follow a lesson plan such as the one outlined above. In an embodiment, a plurality of patients may start out with the same lesson plan. In an embodiment, different patients may be presented with different lesson plans. Among the criteria which may yield different lesson plans are patient age, height, weight, body mass index, and pre-existing conditions, if any.

Along the way, different patients, making different amounts of progress, may have their lesson plans changed. The lesson plan may be tailored to the patient later on, even if the initial lesson plan starts out the same as for other patients. In an embodiment, the lesson regimen may take a predetermined amount of time, for example, 90 days. There may be a plurality of different lessons, for example, 26. In an embodiment, the lessons may be split into two groups. Depending on a patient's progress or reaction to particular lessons, one or more lessons may be repeated one or more times or may be skipped. In an embodiment, depending for example on a patient's background or progress through the nutritional CBT regimen, one or more lessons may be repeated one or more times or may be skipped.

In an embodiment, a practical application of this digitally-based therapy may reside in the ability to draw on a substantial body of patient experience and physiology, involving some or all of the criteria listed earlier. In addition, other aspects of patient history and experience, including prior efforts at controlling or adjusting living, sleeping, and dieting habits, may dictate the tailoring of a lesson plan to a patient.

In an embodiment, lessons may be delivered on a weekly basis. For example, the 26 lessons mentioned above, may be provided over 180 days, approximately one lesson per week. In an embodiment, the lesson selection and sequence for each patient may be the same. The experience with one or more of the lessons may be interactive, wherein the subject may either be prompted for or may volunteer information relevant to the part of the lesson being covered, and may input that information into the digital therapeutic. In an embodiment, a patient's progress through the lessons may be determined by an amount or degree to which the patient may interact during a given lesson. More interaction may promote faster progress from one lesson to another, or skipping of one or more lessons. Less interaction may result in slower progress from one lesson to another, or in repeating of a lesson with less interaction.

In an embodiment, lessons may be accessed on a handheld device such as a phone or tablet, or on a desktop or laptop computer. Different kinds of interactive media may be employed. For example, some lessons may be mainly text. Some lessons may involve audio and/or video recordings. Some or all of the lessons may involve quiz-type questions, to help to ascertain a patient's progress through a particular lesson, or a patient's retention of information imparted during a particular lesson.

In an embodiment, some lessons may include exercises for a patient to perform interactively. For example, a patient may be asked to record information in a journal, either by text or by audio or video recording. The types of information may be physiological in nature (for example, weight, food intake, type and/or amount of exercise, or the like), or may be more personal in nature (for example, reaction to particular types of changes in diet and/or exercise).

In an embodiment, a patient's journaling may be even more personal in nature. For example, a patient may be asked to record feelings about particular lifestyle-related statements. A statement to which a patient somehow expresses a particular level of agreement or affinity may be presented to the patient as an aspect of behavior to adopt or implement.

In an embodiment, one of the exercises may require the patient to select a particular skill module from among a plurality skill modules. That skill module then may become something on which the patient works during the coming week or other time interval between lessons. In an embodiment, the skill module may be an interactive exercise, in which the patient may respond to questions, or may provide evidence of performing the particular skill (for example, posting a picture of the patient performing the skill).

In an embodiment, each lesson may be associated with one or more skills. In an embodiment, as many as five skills may be involved in a particular lesson, and as many as 96 skills over the entire 180 day discipline, but the specific numbers are not critical. In going through a lesson, a patient may select a particular skill on which to work, and may follow through on that work in self-directed fashion throughout the lesson.

In an embodiment, a patient may be asked to set performance goals for a coming period of time, for example, a week, a bi-week, or a month. The performance goals may include specific meal disciplines, for example, type of food, amount, when consumed, and the like. In an embodiment, the performance goals may include exercise minutes, which may be for a particular type of exercise, or may be divided among different types of exercise. Some exercise may be directed toward building cardiac and/or pulmonary endurance. Some exercise may be directed toward building strength and/or flexibility. Some exercise may be directed toward weight loss.

In an embodiment, one or more of the machine learning algorithms in accordance with aspects of the invention may recommend one or more performance goals, based on data relating to similarly-situated patients, including not only patient statistics, but also how often and/or how well similarly-situated patients have met the goals. Goals may relate to meals (frequency, intake amounts, intake types, and the like) and/or exercise minutes, which may include different types of exercise of different intensities for different periods of time, either alone or in combination.

For example, a patient may have poor eating and exercise habits. Part of the overall objective is to improve the patient's eating and exercise habits. Performance goals may be set, or the patient may set the goals themselves, to achieve improvement in diet and exercise. A patient may set fairly low goals. The system may accept the patient's selection without responding, or the system may suggest different, perhaps more aggressive goals in one or both areas, depending on the experience that the machine learning algorithms have accumulated and analyzed for identically, similarly, or comparably situated patients. That experience may relate to goals that such patients have set for themselves, and/or on the goals that such patients actually have been able to achieve. In an embodiment, the system may simply question the patient about their selections, to confirm that the patient is satisfied with the selection, or to allow the patient to choose more aggressive goals. In an embodiment, the system might encourage the patient to choose more aggressive goals, or may simply suggest to the patient that they might choose more aggressive goals. The system's reaction to the patient's selection also may be based, in whole or in part, on the patient's prior selections, the system having taken note of the degree of aggressiveness of the goals that the patient set previously, and the patient's ability to meet those goals.

A patient also may set fairly high or aggressive goals. The system may accept the patient's selection without responding, or the system may suggest different, perhaps less aggressive goals in one or both areas, again depending on the experience that the machine learning algorithms have accumulated and analyzed for identically, similarly, or comparably situated patients. That experience may relate to goals that such patients have set for themselves, and/or on the goals that such patients actually have been able to achieve. In an embodiment, the system may simply question the patient about their selections, to confirm that the patient is satisfied with the selection, or to allow the patient to choose less aggressive goals. In an embodiment, the system might encourage the patient to choose less aggressive goals, or may simply suggest to the patient that they might choose less aggressive goals. The system's reaction to the patient's selection also may be based, in whole or in part, on the patient's prior selections, the system having taken note of the degree of aggressiveness of the goals that the patient set previously, and the patient's ability to meet those goals.

In an embodiment, the lessons presented to a patient as part of an initial algorithm may be an ordered list, and may be the same for all patients. Ordinarily skilled artisans will appreciate that patients may be more likely to fit one profile than another, based on various physiological, personal, cultural, and economic profiles, as well as on such things as geography, access to health care, and other such characteristics as will occur to ordinarily skilled artisans. As the system is able to access more information about patients in all of these categories and more, the system may adapt its approach to intake of new patients, and the initial lesson plan and goal-setting. In an embodiment, the system also may adapt its treatment of existing patients, to the point of moving patients along at different rates depending on patient profile and/or patient performance. As the system is in use to an increasing extent, there may be data generated, for example, to tie performance to treatment effect. It may be possible to implement ever more sophisticated algorithms as a result.

In an embodiment, patients may proceed through the lessons at different rates depending on their progress, and/or on patient perception of how fast they can progress through lessons, how rapidly they can make changes in their diets and other living habits, how quickly they think they can achieve desired goals, and the like.

In an embodiment, the system can may observe how different patients interact with the system. Responsive to those observations, the system may offer different mechanisms to recommend different lesson plans or sequences, or different skills to patients.

In an embodiment, the digital therapeutic provides each patient with a treatment score, which represents real time or near real time feedback about the likelihood of patient success in treatment. In an embodiment, the treatment score is provided using AI/ML techniques, using ground truth data from a large number of patients with many different physical, physiological, biometric, and/or psychological characteristics.

In an embodiment, the treatment score may be computed and updated for each individual. In one aspect, the treatment score may be viewed similarly to a credit score, including ranges. The digital therapeutic may accept all key inputs from the patient, including primary biometrics, and may weight all of the resulting values (for example, using the ground truth data in an AI/ML embodiment) and then convert the resulting data into a score. In an embodiment, the weighting is linear, based on observed versus expected ratios of actions, with some values being more heavily weighted than others. Again, in an embodiment the weighting is informed using AI/ML techniques employing large quantities of ground truth data. In an embodiment, there is sufficient transparency to the algorithm for a patient to understand how their score is calculated, so that the patient can understand what to do to improve their score.

In an embodiment, the digital therapeutic may provide a progress report, showing, for example, changes in biometrics relative to baseline, on a periodic basis. In an embodiment, the progress report is provided on a weekly basis, but other periodicities may be selected as necessary or appropriate. In one aspect, the algorithm may display different content, depending on one or more of the following categories:

Patient is improving and has exceeded targets.

Patient is improving but has not exceeded targets. In an embodiment, the algorithm may suggest that the patient consult additional content, or focus on prior content to help the patient conquer whatever barriers the patient appears to be facing in meeting the particular target or targets. In one aspect, the algorithm may present the patient with additional, perhaps more advanced behavioral strategies.

Patient has not improved, or shows only a little improvement. In an embodiment, the algorithm may give the patient additional guidance, and/or provide encouragement. The algorithm may determine that a less gentle, more direct or harsh approach is warranted. As more data is compiled, there will be room for even more granular messaging.

In an embodiment, the algorithm can vary the treatment plan based on one of the foregoing categories. Where necessary or appropriate, treatment can be augmented to reinforce particular aspects of behavioral therapy pertinent to the particular cardiometabolic disorder, or the patient can be directed toward implementation of more advanced behavioral strategies that were not part of the initial treatment regimen.

In one aspect, the more advanced behavior strategies may be augmentations of the initial treatment regimen. In an embodiment, the augmentations may be personalized to various physiological and/or psychological aspects of the patient, as may other aspects of the treatment regimen. In one aspect, the algorithm's decisions on augmentation may be based just on patient biometrics, working also from the mass of data on similarly situated patients as discussed earlier. In an embodiment, augmentations and other treatment directions may be provided in response to an individual patient treatment score which represents a holistic view of patient health and behavior in response to the regimen. For example, if a patient is not providing sufficiently detailed or frequent reports on different behavioral aspects (for example, meal habits), subsequent treatment could focus on improvement in patient tracking and recording of such data.

In one aspect, it may be possible for the algorithm to select from a plurality of advanced strategies, based on AI/ML feedback from patients with identical or similar relevant profiles.

In an embodiment, possible AI/ML implementations may involve taking advantage of the ability to integrate data from many different sources, for many different patients, to provide the kind of individualized patient guidance that can optimize treatment and promote progress practically, in a way that individual physicians or clinicians cannot. For example, looking at treatment scores of similarly situated patients may make it possible to populate a treatment plan for a particular patient automatically, listing recommendations for actions that would be more likely to provide the best score improvements.

In an embodiment, within the same or different AI/ML algorithms, two or more feedback mechanisms may be employed, either in parallel or in unison, to enable use of treatment scores to provide progress reports and/or other feedback in more granular fashion. For example, a patient might be shown what action(s) or technique(s) seem to be working, and what action(s) or technique(s) might be more likely to improve the patient's biometric data.

In an embodiment, content-directing assessment tools may be employed to take advantage of AI/ML-based feedback. In one aspect, a patient may be asked to respond to survey questions. The patient's answers can be used to prioritize certain lessons or articles, or to tailor feedback, or to direct the patient toward different or more intense training to acquire certain skills.

In an embodiment, as alluded to earlier, particularly robust data sets from patients with a wide variety of physiological and psychological characteristics may enable the AI/ML algorithm to provide predictive analytics.

As an example of the foregoing, the AI/ML system may determine that certain psychological or behavioral characteristics should be given greater weight in determination of or modifications to lesson plans, independent of a patient's particular physiological or biometric characteristics. Alternatively, the AI/ML system may determine that certain physiological or biometric characteristics should be given greater weight in determination of or modifications to lesson plans, independent of a patient's particular psychological or behavioral characteristics.

### EXAMPLES

### Example 1: Behavioral Therapy Through Software-Based Digital Therapeutic

As part of a preliminary investigation into the utility of such an AI/ML implementation, a study was conducted to provide behavioral therapy through a software-based digital therapeutic paired with human support. In the following example, AI/ML techniques were not used. The results of the study are a precursor of the results to be achieved with AI/ML implementations according to aspects of the invention.

Knowledge gained from the use of the digital therapeutic with human support was translated into a software-only digital therapeutic configuration delivering nutritional-CBT. A going-in assumption was that this software-only configuration would improve glycemic control, but to a lower degree than a human +software configuration.

Human support has consisted of remote health coaching delivered telephonically and care escalation to nurses and physicians, as needed. Program effectiveness was studied to improve glycemic control in a non-blinded, single-arm interventional study in 97 adults with type 2 diabetes. After three months, a mean improvement in hemoglobin A1c (HbA1c) of 1% (SD 1.4) was observed in participants with baseline HbA1c >7%. A full description of this study can be found in a peer reviewed article, Berman et al., “Change in Glycemic Control with Use of a Digital Therapeutic in Adults with Type 2 Diabetes: Cohort Study,” JMIR Diabetes 2018:3(1):e4,incorporated by reference herein.

Knowledge gained from the use of the digital therapeutic with human support was translated into a software-only digital therapeutic configuration delivering nutritional-CBT. A going-in assumption was that this software-only configuration would improve glycemic control, but to a lower degree than a human+software configuration.

To determine whether nutritional-CBT could improve glycemic control in patients with type 2 diabetes, data from participants who used a software-only version of the digital therapeutic was examined. Participants were recruited online for usability testing and to provide feedback on content through online surveys. Interested individuals self-identified as having a diagnosis of type 2 diabetes according to any duration, a blood glucose meter at home, and a smartphone. The analysis included participants with a baseline 3-day average fasting blood glucose (FBG)>152 mg/dL; a value corresponding to an hemoglobin HbA1c>7%. 74 adult participants were identified for inclusion. Table 2 below shows baseline characteristics of the participants.

TABLE 2

| Number of participants | 74 | |
|---|---|---|
| Age, years, mean (SD) | 55.2 | (7.0) |
| Body Mass Index, kg/m², mean (SD) | 34.7 | (7.9) |
| Glycemic medications reported, count, mean | 2.2 | (0.9) |
| (SD) | | |
| Fasting blood glucose, 3-day average, mg/dL, | 199.4 | (45.1) |
| mean (SD) | | |
| Estimated HbA1c, %, mean (SD) | 8.7% | (2.0) |
| Time since diabetes diagnosis, years, mean | 10.6 | (7.1) |
| (SD) | | |
| Gender, % female (n) | 72% | (53) |

| | |
|---|---|
| Geographic location, U.S. states represented, | 32 |
| count | |

As part of typical use over 90 days, participants were encouraged to self-monitor their fasting blood glucose daily with a home glucometer and to enter those values directly into the app. In addition to interactive nutritional-CBT content, weekly goal-setting was used to guide participants in replacing highly-processed foods with whole foods, and steadily increase the proportion of meals coming mostly from plants. In the digital therapeutic, this was referred to as eating more “plant-based meals”.

Changes in self-reported FBG were examined by looking at the difference between 3-day averages anchored by the first and last values reported. To examine whether participants who were not tracking FBG might negatively impact population outcomes, an intent-to-treat (ITT) analysis was performed by assuming no change in FBG for those who did not track after reporting a baseline.

Mean change in FBG was −22.9 mg/dl (SD 42.0) over an average of 69 days (SD 30.0), which corresponds to an estimated HbA1c change of −1%. Table 3 below shows changes in FBG.

TABLE 3

| | | Fasting Blood Glucose, mean (SD) | | |
|---|---|---|---|---|
| Participant Sub- | | Baseline | | |
| group | n | Average | Last Average | Change |
| All | 74 | 199.4 (45.1) | 176.5 (49.8) | −22.9 (42.0) * |
| Stable glycemic | 65 | 197.7 (43.8) | 176.0 (49.2) | −21.7 (40.2) * |
| Baseline HbA1c | 65 | 187.4 (32.6) | 168.4 (45.1) | −19.0 (40.1) * |
| 7-11% | | | | |

* Paired t-test comparing baseline average to most recent average, p < 0.001

In the study, a non-AI/ML version of the digital therapeutic was used in a real-world setting while participants continued usual medical care. Participants were free to change medications as directed by their physician and were encouraged to adhere to prescribed medications and report any changes within the app. The impact of changes to glycemic medications while using the app was examined. At baseline, 93% (69/74) of participants reported using anti-hyperglycemic medications. Over the 90-days observed, nine of the 69 participants reported a change (dose or count of meds); two reported a decrease and seven reported an increase. Exclusion of these nine participants from the analysis did not have a meaningful effect on the mean improvements in FBG observed.

It was considered desirable to examine whether outliers might overly influence outcomes and to align with the inclusion criteria for an upcoming randomized controlled trial. Accordingly, the mean change in FBG in participants with an estimated baseline HbA1c between 7 and 11% was examined. In these 65 participants, the mean change in FBG was −19.0 mg/dL over a mean of 71 days (SD 27). Using their ending 3-day FBG average, 43% (28/65) of participants in this subset were now at goal (FBG<152 mg/dL, estimated HbA1c<7%), and 19% (12/65) met a much more aggressive goal for glycemic control (FBG<130 mg/dL, estimated HbA1c<6.5%).

Relationships between improvements in glycemic control and use of key nutritional-CBT features (therapy lessons, skills, and weekly goal setting) and self-reported diet and exercise behaviors were explored. Counts were summed for each participant and changes in FBG were examined for thirds of the entire population, from lowest to highest (tertiles) of feature or behavior type. In regression models, baseline average FBG, years since diagnosis of diabetes, and the duration of observation (the length of time between first and last value reported) were controlled for.

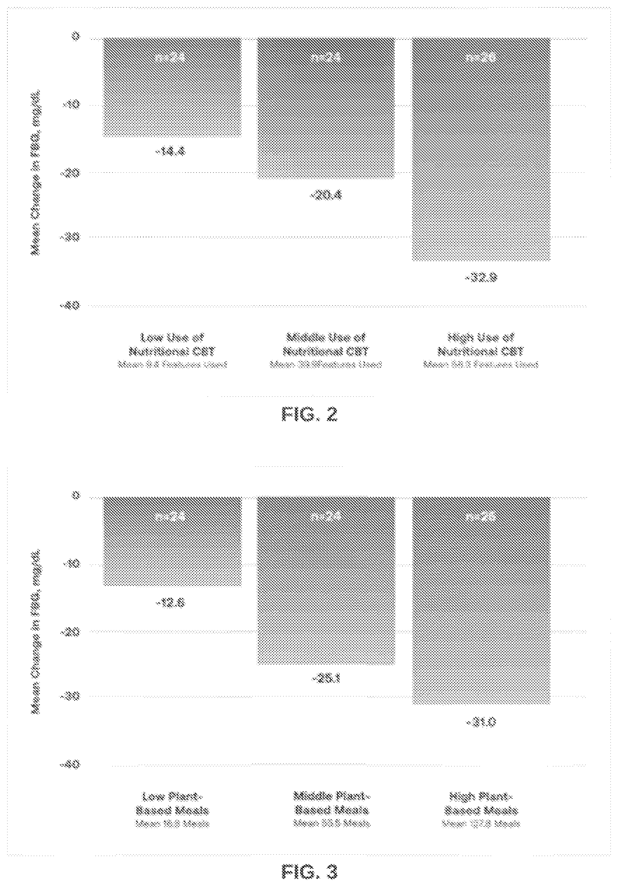

Of all variables explored, the use of nutritional-CBT content and the number of plant-based meals tracked demonstrated the strongest dose responses with improvements in FBG (FIGS. 2 and 3). In addition, a significant positive correlation was found between the use of the nutritional-CBT and change in dietary pattern (p<0.05 using linear regression).

In FIG. 2, the mean sum of nutritional-CBT actions completed for tertiles low to high were 9.4, 29.9 and 58.3. Pairwise comparison of the least square means controlling for baseline FBG, years since diagnosis and duration of FBG tracking was used to compare FBG across tertiles: low vs high tertile p=0.06, middle vs high tertile p=0.04.

In FIG. 3, the mean sum of plant-based meals reported for tertiles low to high were 17, 56 and 128. Pairwise comparison of the least square means controlling for baseline FBG, years since diagnosis and duration FBG tracked was used to compare FBG across tertiles: low vs high tertile p=0.18, middle vs high tertile p=0.46.

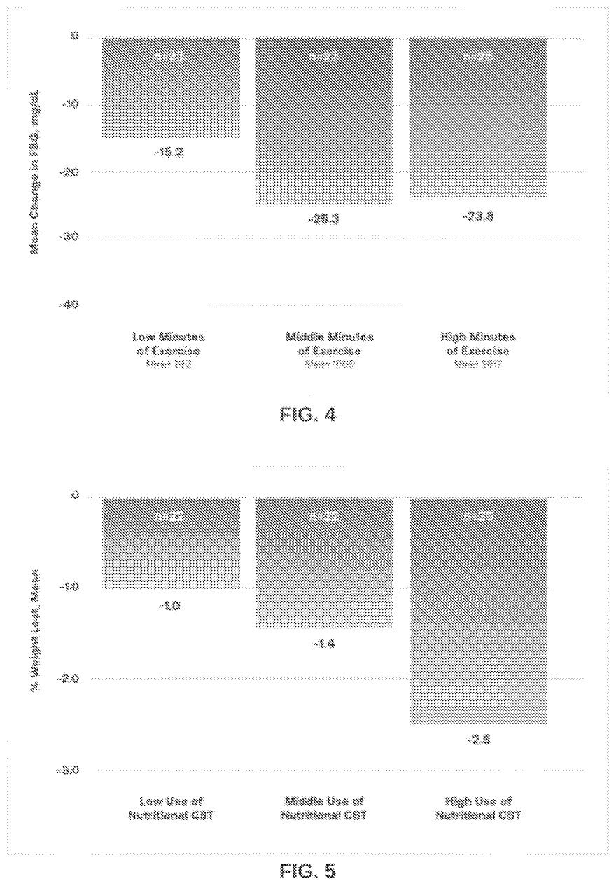

In the results, as seen for example in FIG. 4, it is possible to observe a trend between increased minutes of exercise reported and improvements in FBG. However, the dose-response pattern was not as clear as the pattern seen with nutritional-CBT and changes in diet.

FIG. 4 shows changes in FBG by tertile of minutes of exercise. In the bar chart of FIG. 4, mean minutes of total exercise over the 90 days reported for tertiles low to high were 262, 1,000 and 2,617. Pairwise comparison of the least square means controlling for baseline FBG, minutes of exercise in week 1, years since diagnosis and duration FBG tracked was used to compare FBG across tertiles: low vs high tertile p=0.08, middle vs high tertile p=0.48.

Weight loss also can lead to large improvements in glycemic control if participants lose 5-7% of body weight. The study explored whether changes in weight were associated with improvements in glycemic control. An average weight loss of 1.7% (SD 2.2) over a mean of 61 days was found between 3-day averages for baseline and last weight values reported. In a linear regression model, percent weight loss was significantly correlated with improvements in FBG (p=0.01). FIG. 5 shows a tertile analysis of nutritional-CBT use and changes in weight. The bar chart indicates that higher use may be associated with larger improvements in weight. There appeared to be greater weight loss in patients who used nutritional-CBT more extensively, but the degree of weight loss observed suggests that weight loss may not have been the major driver of glycemic changes.

FIG. 5 shows percent weight loss by tertile of nutritional CBT use. In FIG. 5, pairwise comparison of the least square means controlling for baseline FBG, years since diagnosis and duration FBG tracked was used to compare FBG across tertiles: low vs high tertile p=0.02, middle vs high tertile p=0.09.

In the study, usage data were examined to understand patterns of engagement, including frequency of use, time spent in each session of use, and duration of engagement. On average, participants used the app 4.5 days a week and spent 5 minutes in the app each day they used it. The duration of engagement was determined by the interval from the first day of use to the last day a participant either completed a nutritional-CBT task or entered a biometric value. 72% of the participants engaged with the program for 10 weeks or longer.

Participants were sent an online survey 10 weeks after starting their use of the app. 51% (38/74) completed the survey. The survey included questions about their experience using the app and asked about whether they would recommend the program to a friend or family member with diabetes. This question is referred to as a standardized Net Promoter Score (NPS) question. A calculated NPS score of 0 to 30 is considered ‘good’, 30 to 70 is ‘great’ and over 70 is ‘excellent’. The average score for the healthcare industry in a recent report was 27. The calculated NPS score here was 58, which is “great”.

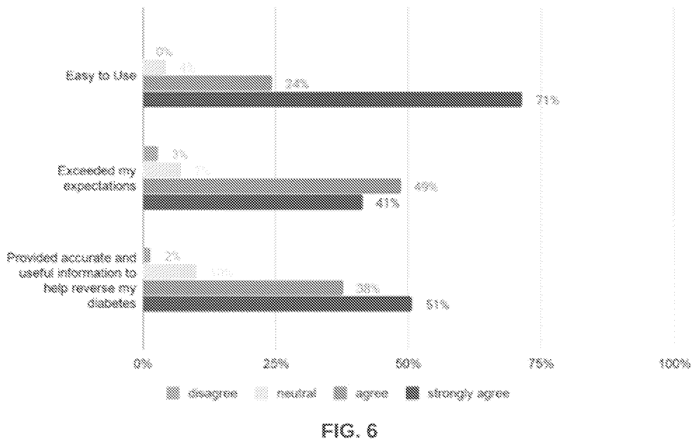

FIG. 6 indicates that overall participant opinions were positive in response to questions about ease of use, relevance of content, and ability to meet or exceed expectations.

The just-discussed study regarding a digital therapeutic delivering Nutritional-CBT resulted in clinically meaningful improvement in glycemic control. The mean decrease in FBG of −22.9 mg/dL corresponds to approximately a 1% reduction in HbA1c. An HbA1c reduction of 1% has been associated with a 21% decrease in diabetes related mortality and a 40% reduction in microvascular complications in the UK Prospective Diabetes Study with long-term follow up. This data is evidence that a software-only digital therapeutic in accordance with aspects of the present invention can serve as a standalone treatment.

The study results indicate a significant dose response between the degree of engagement in nutritional-CBT and improvements in glycemic control among adults with type 2 diabetes. This result is encouraging because it indicates that digitally-delivered behavioral therapy using only software has the potential to treat disease at scale.

Reductions in blood glucose were more significant and occurred faster than expected. Software-based treatment in accordance with aspects of the present invention can enable patients to make behavioral changes at a gradual pace. But the study results revealed more rapid blood sugar control than expected, with 38% of participants achieving a fasting blood glucose level less than 152 mg/dL (corresponding to an HbA1c<7%, which is commonly regarded as the goal for HbA1c for most patients with type 2 diabetes) and 16% achieving a fasting blood glucose less than 130 mg/dL (corresponding, on average to an HbA1c<6.5%, a much more aggressive goal for HbA1c) after an average of 69 days.

The foregoing results indicate that use of the software-based treatment in accordance with aspects of the invention can result in even greater improvements.

Improvements in blood glucose occurred in participants from across the country and with long standing diabetes. One hypothesis has been that only newly diagnosed patients will benefit from behavioral therapy. However, the study results indicate a strong efficacy signal in patients who were on average diagnosed with diabetes more than 10 years ago. The study also had excellent geographic diversity with participants from 32 states, including those with increasing prevalence of diabetes (e.g., Florida, Indiana and North Carolina).

According to the Centers for Disease Control (CDC), older individuals, especially those with diabetes and heart disease, are at higher risk of serious complications from COVID-19. Validated digital therapeutics can play an important role by being rapidly deployed into large populations to improve health, remotely monitor disease biometrics and self-reported symptoms, and escalate care as needed for triage and medical direction. This may be an effective way to protect the most vulnerable high-risk patients and keep them out of hospitals, emergency rooms and primary care clinics, alleviating burden on the system and associated costs.

When healthcare resources become severely stressed, and when healthcare resources simply are not readily available onsite to patients in remote areas, software-based treatment in accordance with aspects of the present invention can support and improve the health of patients with diabetes and other cardiometabolic conditions. This work highlights the potential for using digital therapeutics, once validated, to advance the care of these patients without increasing demand on the health system.

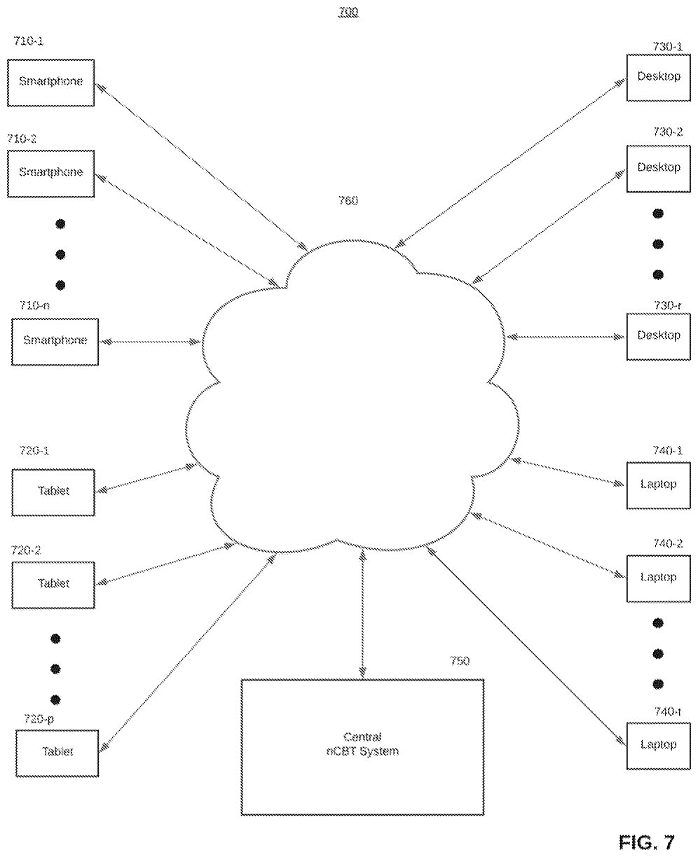

FIG. 7 shows, at a high level, aspects of a nutritional cognitive behavior therapy (nCBT) system 700 according to an embodiment, to illustrate aspects of system operation. A plurality of smartphones 710-1, . . . 710-n, tablets 720-1, . . . 720-p, desktops 730-1, . . . 730-r, and laptops 740-1, . . . , 740-t may communicate with a central nCBT system 750 to receive, provide, or exchange information as previously described. The smartphones 710-1, . . . 710-n, tablets 720-1, . . . 720-p, desktops 730-1, . . . 730-r, and laptops 740-1, . . . , 740-t may communicate with central nCBT system 750 either directly or through a network or cloud 760.

Depending on the embodiment, subjects may use a respective one (or, in some instances, more than one) of the smartphones 710-1, . . . 710-n, tablets 720-1, . . . 720-p, desktops 730-1, . . . 730-r, and laptops 740-1, . . . , 740-t to download a digital therapeutic either directly from central nCBT system 750, or through a service in or connected to the network or cloud 760. Subjects also may use a respective one or more of smartphones 710-1, . . . 710-n, tablets 720-1, . . . 720-p, desktops 730-1, . . . 730-r, and laptops 740-1, . . . , 740-t to receive therapy lessons and/or skill-building exercises either one at a time, or more than one at a time, from central nCBT system 750. Subjects may provide responses, biometric data, and the like to central nCBT system 750 through respective ones of smartphones 710-1, . . . 710-n, tablets 720-1, . . . 720-p, desktops 730-1, . . . 730-r, and laptops 740-1, . . . , 740-t. Central nCBT system 750 may in turn may generate one or more personalized notifications, including but not limited to guidance, progress reporting, treatment score, reminders, nudges, and rewards, to subjects who receive these via respective ones of smartphones 710-1, . . . 710-n, tablets 720-1, . . . 720-p, desktops 730-1, . . . 730-r, and laptops 740-1, . . . , 740-t. In an embodiment, responsive to biometric data from subjects, central nCBT system 750 may provide biometric notifications, including indications of danger levels.

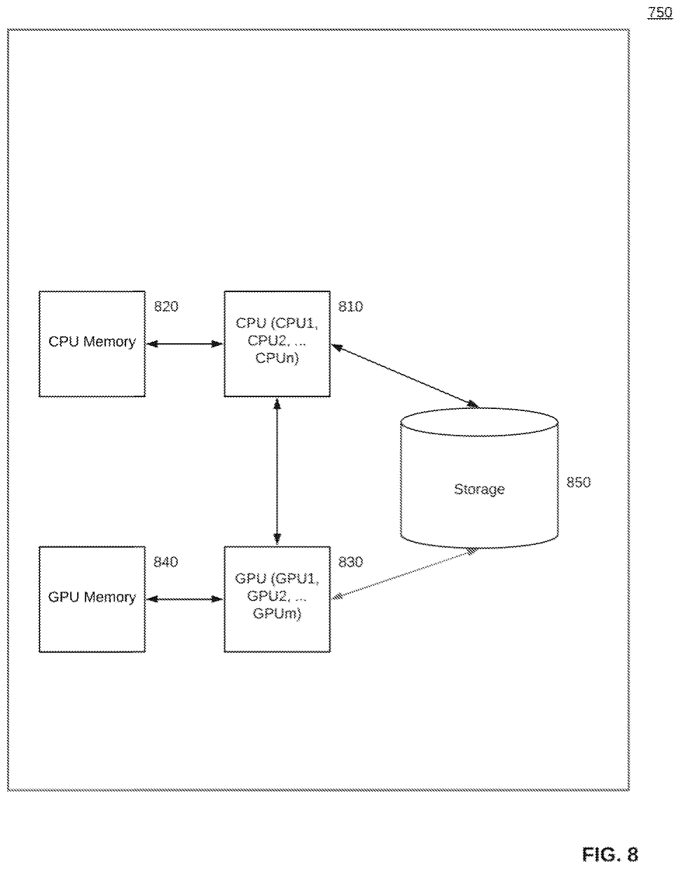

FIG. 8 shows aspects of the central nCBT system 750 according to an embodiment. It should be noted that, depending on the embodiment, there may be multiple instances of central nCBT system 750 distributed in different locations, all communicating with each other via the cloud 760 and, in some instances, communicating with different subjects via respective ones of smartphones 710-1, . . . 710-n, tablets 720-1, . . . 720-p, desktops 730-1, . . . 730-r, and laptops 740-1, . . . , 740-t. In an embodiment, a subject who is traveling may access a nearest or most convenient one of the multiple instances of central nCBT system 750. In such a configuration, data particular to the user is stored either centrally for the various central nCBT systems 750 to access, or locally in each of the various central nCBT systems 750. In the case of such local storage, updated information for respective subjects may be provided periodically to each of the various central nCBT systems 750.

An exemplary central nCBT system 750 may include one or more central processing units (CPUs) 810, each associated with CPU memory 820. Depending on the embodiment, each CPU 810 may have its own associated CPU memory 820. Alternatively, the CPUs 810 may share the CPU memory 820. Depending on the embodiment, one or more of the CPUs 810 may communicate with each other over a bus (not shown), to which CPU memory 820 also may be connected. In embodiments, CPU memory 820 may include volatile and/or non-volatile memory, and in some instances, non-transitory storage.

An exemplary central nCBT system 750 also may include one or more graphics processing units (GPUs) 830, each associated with GPU memory 840. Depending on the embodiment, each GPU 830 may have its own associated GPU memory 840. Alternatively, the GPUs 830 may share the GPU memory 840. Depending on the embodiment, one or more of the GPUs 830 may communicate with each other either directly or over a bus (not shown), to which GPU memory 840 also may be connected. In embodiments, GPU memory 840 may include volatile and/or non-volatile memory, and in some instances, non-transitory storage. Depending on the embodiment, one or more GPUs 830 may communicate with one or more CPUs 810 either directly over a bus (not shown).

Storage 850 may take different forms, from one or more hard disk drives (HDD) to one or more solid state drives (SSD), to combinations of one or more HDD and one or more SSD.

Depending on the embodiment, one or more of the machine learning algorithms discussed above may reside in one or more GPUs 830, depending on the algorithm and its associated hardware requirements.

### Example 2: Pivotal Clinical Trial

Particularly relevant aspects of the trial discussed in this Example included the use of a nationally representative, diverse patient population (Table 4); use of investigators that mirror real-world prescribers; and robust study design employed to minimize bias and set a high comparison bar. For example, the control arm used in the study is Standard of Care (SOC) (i.e., gold standard care), not just treatment per usual; medication use and adjustment by investigators was not limited, only prandial insulin was excluded; and patients were not mandated nor incentivized to use the disclosed digital therapeutic, instead they were free to self-select dose.

TABLE 4
Patient population

| | | Standard | Digital |
|---|---|---|---|
| | | of Care | Therapeutic |
| Parameter/Category | Statistic | (n = 343) | (n = 325) |
| Age (yrs) | mean | 58.1 | 58 |
| % Female | % | 56.3 | 56 |
| Race | % | | |
| White | | 61.2 | 62.2 |
| Black or African | | 29.2 | 29.5 |
| American | | | |
| Asian | | 5.2 | 5.2 |
| American Indian or | | 1.7 | 1.2 |
| Alaskan Native | | | |
| Native Hawaiian or | | 0.6 | 0.3 |
| other Pacific Islander | | | |
| Ethnicity - Hispanic | % | 14 | 17.2 |
| or Latino | | | |
| Median Household | mean | $67,737 | $69,789 |
| income by ZIP code | | | |
| % High school degree | % | 42% | 38.2 |
| or some college but | | | |
| no degree | | | |

As seen at Table 4, the study included a nationally representative, diverse patient population. The population was recruited from 6 states, and included groups underrepresented in clinical trials, with historically poor access to care.

Participants had long-standing type 2 diabetes, high cardiovascular risk, multiple comorbidities, and extensive medication use (Table 5).

TABLE 5
Patient population medical details

| | | Standard | Digital |
|---|---|---|---|
| | | of Care | Therapeutic |
| Parameter/Category | Statistic | (n = 343) | (n = 326) |
| BMI (kg/m2) | mean | 34.7 | 34.6 |
| Baseline HbA1c (%) | mean | 8.1 | 8.2 |
| Years since diagnosis | mean | 10.9 | 11.0 |
| % on 2 or more | % | 67.5 | 68.4 |
| Antihyperglycemic | | | |
| medications | | | |
| Using | % | 71.7 | 67.1 |
| antihypertensive | | | |
| medications | | | |
| % on 2 or more | % | 66.5 | 69.0 |
| number of | | | |
| antihypertensive | | | |
| medications (for those | | | |
| treated for | | | |
| hypertensions, 67%% | | | |
| of participants) | | | |
| 10 year CV Risk score | mean | 15.1 | 15.1 |
| Number of | mean | 2.7 | 2.8 |
| Comorbidities | | | |

Baseline diabetes medications reveal robust background therapy compared with general diabetes population (Table 6).

TABLE 6
Background therapies

| | General Diabetes | Standard | Digital |
|---|---|---|---|
| Medication Class | Population (2018)¹ | or care | Therapeutic |
| All | 82.7% | 96.5% | 96.0% |
| Metformin | 59.5% | 79.9% | 80.1% |
| Sulfonylureas | 24.4% | 34.4% | 36.2% |
| SGLT2 inhibitors | <7.1% | 24.8% | 21.8% |
| GLP-1 analogues | <7.1% | 24.8% | 19.0% |
| Insulin | 25.6% | 19.5% | 17.8% |
| DPP-4 inhibitors | 10.8% | 13.7% | 17.2% |
| Thiazolidinediones | 3.3% | 5.0% | 5.5% |
| Meglitinides | — | 0.3% | 0.9% |

¹Fang et al. Trends in Diabetes Treatment and Control in U.S. Adults, 1999-2018. N Engl J Med 2021: 384; 2219-2228

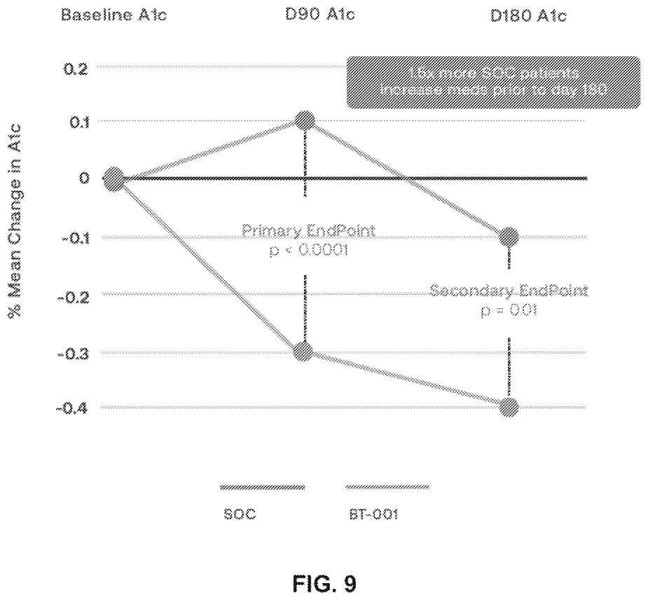

As shown in FIG. 9, the disclosed digital therapeutic (BT-001) demonstrated sustained and improved response at 180 days, with absolute A1c reduction advancing from 0.3% to 0.4%. The disclosed digital therapeutic reduced A1c despite on-study addition of more diabetes medication in the SOC control group. Notably, both primary (A1c between group delta—0.4%, p<0001) and secondary endpoints (A1c delta—0.3%, p.01) were met. Half of patients in the test arm achieved clinically meaningful changes with absolute mean A1c reduction of 1.3% (SD 0.8%) in this subgroup. The study showed robust safety data, with significantly fewer adverse events in the test arm (p<0.001). Digital therapeutic use was associated with multiple additional cardiometabolic benefits and lower medication and lower healthcare utilization.

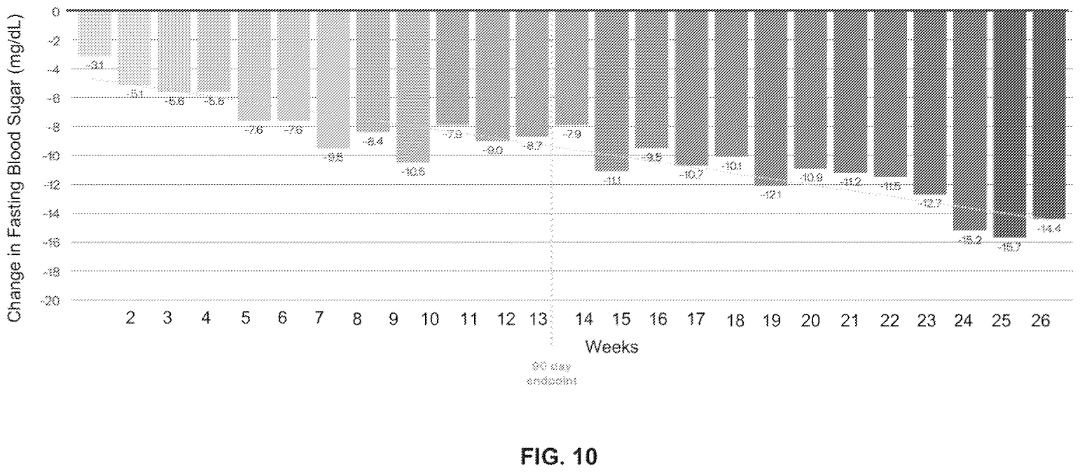

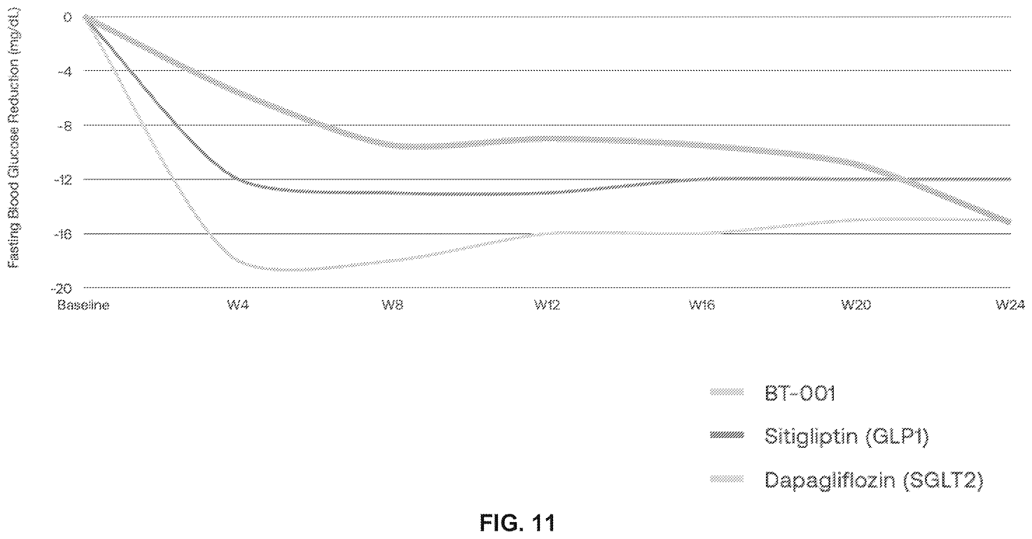

The trending average change in fasting blood glucose shows gradual and steady improvements, with no clear peak (FIG. 10). Shown at FIG. 11 is a graph illustrating trends in fasting blood glucose in different therapies (BT-001), Sitigliptin (GLP1), or Dapagliflozin (SGLT2) (see Ferrannini et al., Diabetes Care. (2010); 33: 2217-2224); Goldstein et al., Diabetes Care. (2007); 30(8): 1979-1987). It is to be understood that the data at FIG. 11 is from different studies with different trial designs and patient populations.

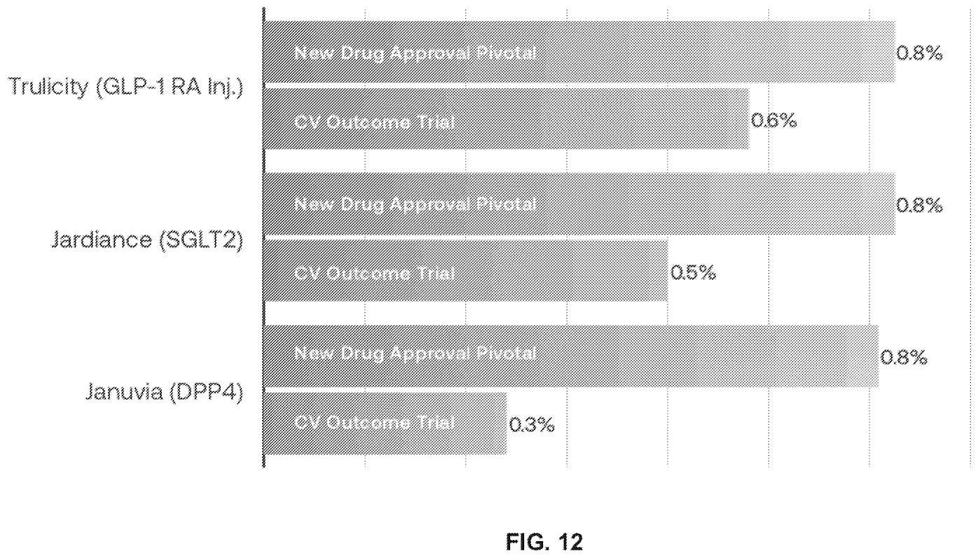

Turning to FIG. 12, depicted is a graph illustrating that cardiovascular outcome trials (CVOTs) show lower relative A1c reduction compared with new drug pivotal for same drug (Gerstein et al., Lancet. (2019); 394(10193): 121-130; Umpierrez et al., Diabetes Care. (2014); 37(8):2168-2176; Zinman et al., New England J of Medicine. (2015); 373(22): 2117-2128; Roden et al., Lancet Diabetes Endocrinol. (2013); 1(3): 208-219; Green et al., The New England J of Medicine. (2015); 373(3): 232-242; Aschner et al., Diabetes Care. (2006); 29(12): 2632-2637). Hence, trial design may influence A1c reduction observed. The disclosed digital therapy pivotal trial design is more similar to diabetes cardiovascular outcome trials (Table 7).

TABLE 7
Digital therapy trial design comparison

| | | | Digital |
|---|---|---|---|
| | | | Therapeutic |
| Trial | New drug | CV outcome | pivotal |
| Characteristic | pivotal | trial | (BT-001) |
| Poorly controlled | Yes | Yes | Yes |
| diabetes | | | |
| at baseline | | | |
| Diabetes disease | <10 years | >10 years | >10 years |
| duration (mean) | | | |
| Baseline Therapy | Highly (e.g., | Moderately (e.g., | Minimally (Only |
| Limited | Metformin | max of 2 drugs | prandial insulin |
| | monotherapy) | at baseline) | excluded) |
| Comparison Arm | Placebo or | Standard of care | Standard of care |
| | single agent | | |
| Dosage of | Yes | Yes | No |
| Investigational | | | |
| Therapy | | | |
| Controlled | | | |

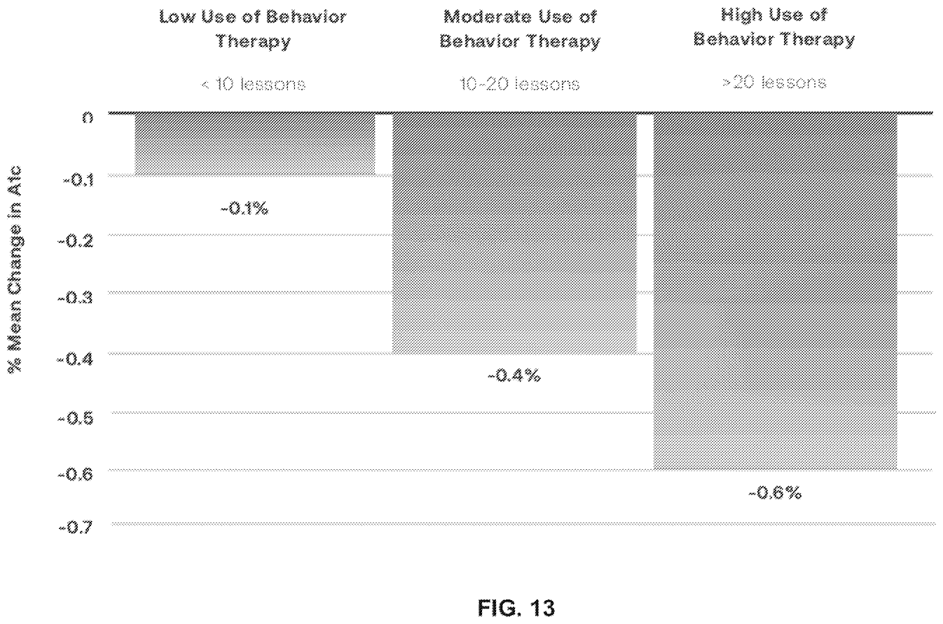

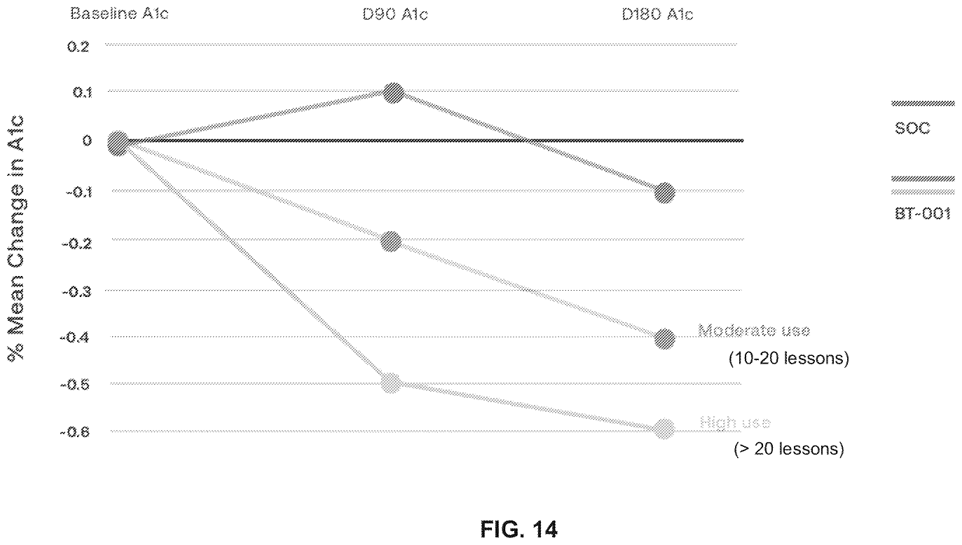

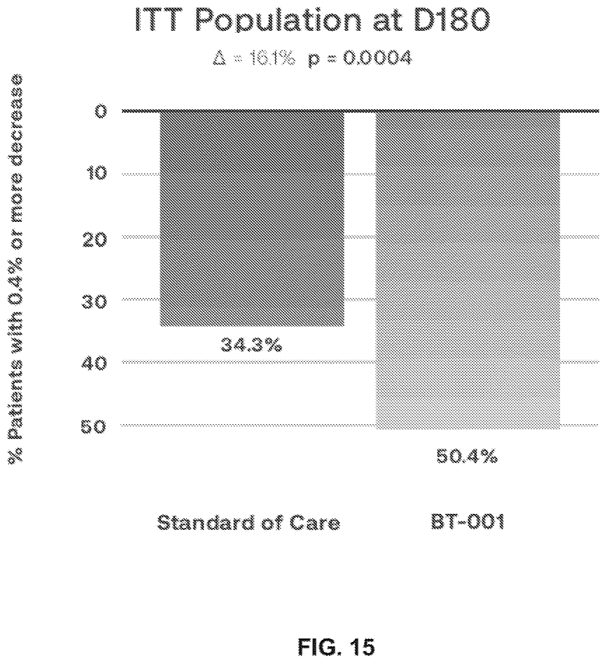

Patients were instructed to self-select dose of nCBT. Higher dose of nCBT lessons completed was associated with larger A1c improvements at 180 days (FIG. 13). The higher dose subgroup (>20 lessons) showed substantially greater A1c improvement compared to SOC control group (FIG. 14). 1.5× more digital therapy patients achieved meaningful A1c change. Significant improvements were observed in the test group despite the use of fewer diabetes medications (FIG. 15). Notably, 30% of patients achieved a 1% or more A1c reduction vs. 17% for control group, p=0.001), and 30% of patients achieved blood sugar control target of A1c<7% vs. 20% SOC, p=0.009)

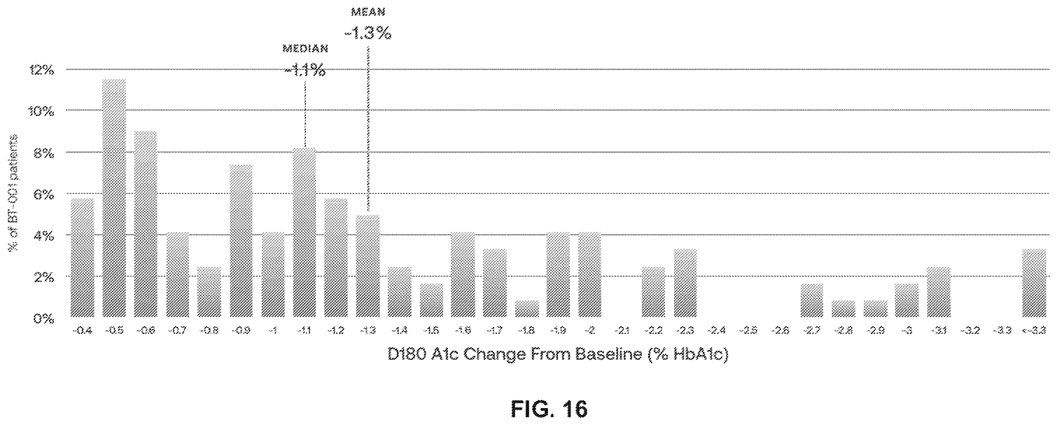

In the study, meaningful responders, defined as 0.4% or more A1c improvement, show a range of large improvements at 180 days (FIG. 16). Furthermore, 180 day safety data indicated significantly fewer adverse events (AEs) and fewer serious AEs (SAEs) (Table 8).

TABLE 8
180 day safety data

| # subjects who | SOC (n = 343) | | | BT-001 (n = 325) | | | |
|---|---|---|---|---|---|---|---|
| experienced | Subjects n (%) | | Events n | Subjects n (%) | | Events n | |
| AE | 188 | (54.8%) | 324 | 135 | (41.5%) | 265 | (p < 0.001) |
| Serious AE | 24 | (7.0%) | 26 | 9 | (2.8%) | 9 | (p = 0.01) |

| AE possibly or | 0 | (0%) | 0 | 3 | (0.9%) | 4 |
|---|---|---|---|---|---|---|
| probably related | | | | | | |
| to study | | | | | | |
| intervention | | | | | | |
| AE related to | 0 | (0%) | 0 | 0 | (0.0%) | 0 |

medical software

Patients subjected to the disclosed digital therapy avoided more serious AEs commonly found in type 2 diabetes (Table 9).

TABLE 9
Patients on digital therapy avoidance of serious AEs

| | SOC (n = 343) | | BT-001 (n = 325) | |
|---|---|---|---|---|
| # patients who | Subjects | | Subjects | |
| experienced | n (%) | Events n | n (%) | Events n |
| SAE | 24 (7.0%) | 26 | 9 (2.8%) | 9 |
| SAE possibly | 14 (4.1%) | 14 | 5 (1.5%) | 5 |
| related to | | | | |
| diabetes/ | | | | |
| cardiometabolic | | | | |
| Cardiovascular | 6 (1.7%) | 6 | 2 (0.6%) | 2 |
| Respiratory | 2 (0.6%) | 2 | 1 (0.3%) | 1 |
| Infectious | 6 (1.7%) | 6 | 2 (0.6%) | 2 |
| Other SAEs | 12 (3.5%) | 12 | 4 (1.5%) | 4 |
| Death | 1 (0.3%) | 1 | 0 (0.0%) | 0 |

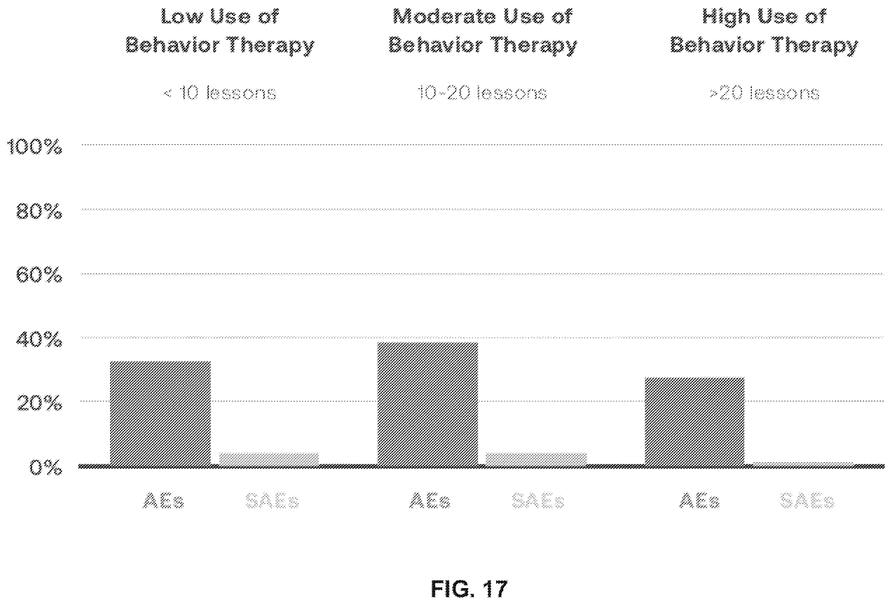

Advantageously, higher digital therapy dose was found to be associated with larger improvements, but not higher rates of AEs (FIG. 17).

Generally speaking, the safety profiles of top-performing diabetes drugs differ from the disclosed digital therapy (Table 10).

TABLE 10
Safety profiles

| Adverse Reaction | | | Digital Therapeutic |
|---|---|---|---|
| (>/=5%) | GLP1 | SGLT2 | (BT-001) |
| Nausea | Yes | No | No |
| Vomiting | Yes | No | No |
| Diarrhea | Yes | No | No |
| Abdominal Pain | Yes | No | No |
| Constipation | Yes | No | No |
| Female genital | No | Yes | No |
| mycotic infections | | | |
| Urinary track | No | Yes | No |
| infections | | | |
| Device related | N/A | N/A | <1% |
| adverse events | | | |

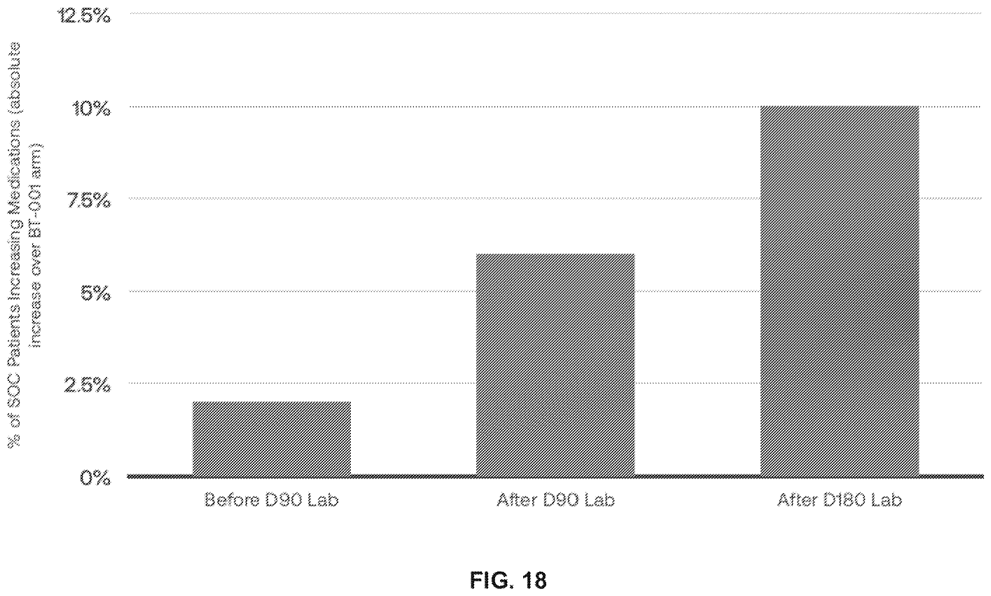

Antihyperglycemic medication utilization and healthcare utilization increased more in SOC control group with a widening gap over six months (FIG. 18). Patients relying on the disclosed digital therapy experienced fewer hospitalizations, ER visits, and outpatient visits over length of study.

During 180 days of use, patient engagement and persistence exceeded benchmarks for consumer and health wellness apps (Apptentive 2022 Mobile Customer Engagement Benchmark Report. % Retention at 90 days). Retention was found to be 94% in the group for which the disclosed digital therapy was tested. After 180 days, 81% of the patients were using the app, average minutes spent per day on the app was about 5.9 minutes, and the NPS score after 180 days was 61.

### Example 3: LivVita Study

Both type 2 diabetes and hypertension have similar underlying metabolic etiologies to Metabolic dysfunction-associated steatotic liver disease (MASLD), including obesity, insulin resistance, and chronic meta-inflammation, which are directly related to healthy lifestyle behaviors. Enrollment in a LivVita liver study for nonalcoholic fatty liver disease (NAFLD) and nonalcoholic steatohepatitis (NASH) was completed. NAFLD/NASH affects over 64 million adults in the U.S., resulting in over $100 billion in direct healthcare costs annually. There are currently no FDA approved therapeutics for treating NASH/NAFLD.

The clinical study evaluated the feasibility of nCBT to reduce liver fat and improve liver disease biomarkers as a potential treatment for fatty liver disease. This single arm interventional cohort study has completed enrollment of 22 adult patients from two specialized liver treatment clinics with data expected Q4 of 2022. The primary objective was to evaluate the feasibility and efficacy of nCBT in improving liver health in patients diagnosed NAFLD/NASH. The secondary objective was to gather user-experience feedback that will be used to improve usability of nCBT for future use in patients with NAFLD/NASH and evaluate intervention safety in this patient population. In addition, the study explored the degree to which various non-invasive imaging technologies, composite scores, and serum laboratory biomarkers are sensitive to behavioral changes induced by nCBT.

Regarding changes in nCBT content for the LivVita study, no major changes were made to the product experience. Minimal changes were made to content knowing that additional changes can be made if needed prior to full a pilot or pivotal trial. No significant changes to skills or goal setting were made.

### Methods

This prospective, open-label study was conducted at two hepatology clinics. Eligible patients had a baseline FibroScan controlled attenuation parameter >274 dB/m. Participants were given access to a PDT containing a novel form of cognitive behavioral therapy designed to treat cardiometabolic disease. Laboratory assessments, FibroScan, and magnetic resonance imaging proton density fat fraction (MRI-PDFF) imaging were conducted preintervention and postintervention.

Participants were recruited through the clinics' existing patient population. Interested participants were screened for eligibility by medical history, baseline labs, biometrics, and focused physical exam. The study included 18-75-year-olds with a diagnosis of MASLD or metabolic dysfunction-associated steatohepatitis (MASH), confirmed by screening FibroScan (Echosens, Paris, France) controlled attenuation parameter (CAP) score >274 dB/m, with body mass index ±30 kg/m2, and possession of smartphone (iPhone or Android) capable of running the PDT. Exclusion criteria included an inability to read or comprehend English (the PDT is only currently available in English), recent history of alcohol or substance abuse, or a history of other liver diseases. Those who met initial eligibility requirements received a baseline magnetic resonance imaging proton density fat fraction (MRI-PDFF) to further assess liver fat accumulation.

Eligible participants completed a call where they had the opportunity to ask questions before beginning the intervention. Participants were then sent a link to create an account and download the PDT from their smartphone app store. Treatment started after completion of a brief automated onboarding after which participants used the PDT at will. After 90 days, participants returned to the clinic to repeat the physical exam, biometrics, labs, FibroScan, and repeated the MRI-PDFF.

The primary outcome was the mean change in percent liver fat from baseline to 90 days for the subset of participants with an elevated baseline MRI-PDFF reading, defined as ,â·10%.

The mean change from baseline in percent liver fat at day 90 was assessed in all enrolled subjects as a secondary endpoint. Other indicators of liver health, including alanine transaminase (ALT), CAP score, Fast score, FIB-4 index, and weight change, were also explored in all participants, regardless of baseline PDFF measurement. Change in MASH risk from baseline to end of intervention was assessed using change in Fast score categories (>0.67=high risk for MASH, 0.67-0.35=indeterminate, <0.35=MASH is unlikely).

### Results

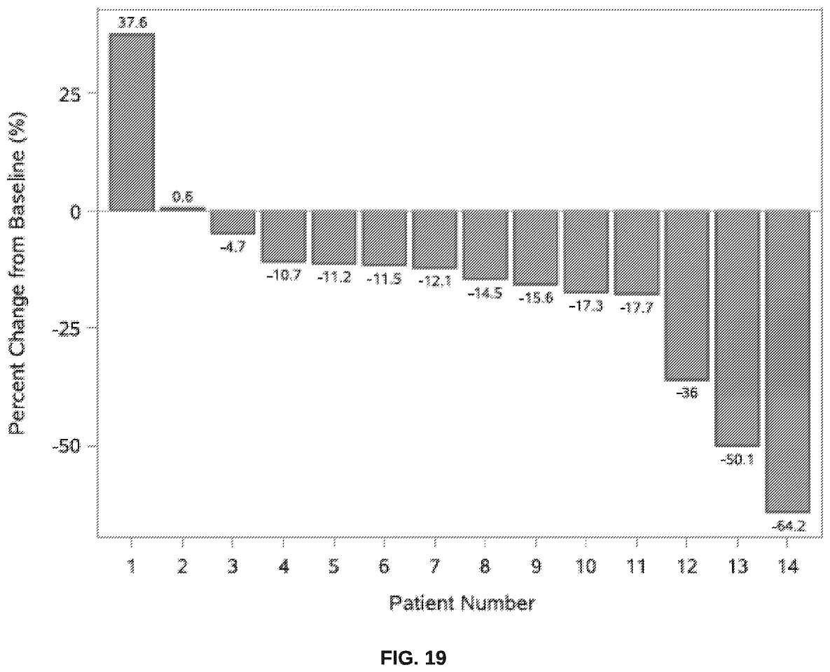

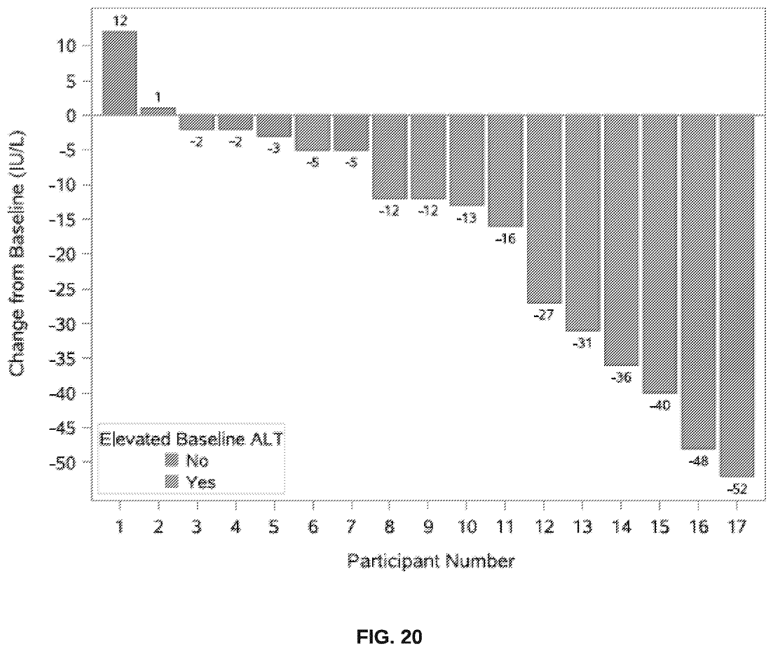

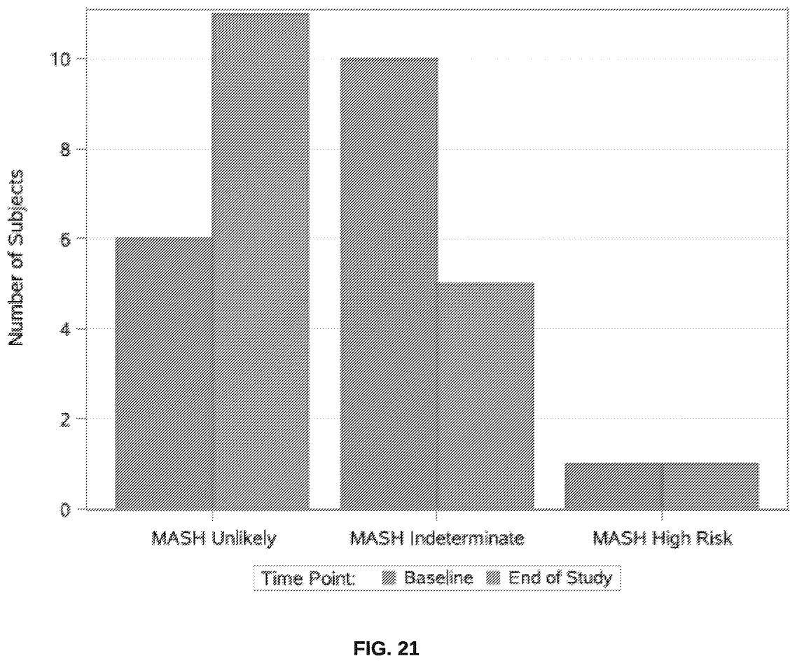

Twenty-two participants were enrolled. Mean baseline fat fraction on MRI-PDFF was 18.7%. After the 90-day intervention, the mean relative reduction in MRI-PDFF was −16.2% (P=0.011) in those with baseline PDFF ,â·10% (FIG. 19). The maximum change observed within a single participant was a −64.2% relative reduction in PDFF from baseline. FIG. 20 shows the change in ALT for each participant. The mean change in ALT was −17.1 IU/L (±18.6, n=17, P=0.002). In those with an elevated ALT at baseline (ALT >19 IU/L for females and >30 IU/L for males), the mean reduction was 22.5 IU/L (±17.6, n=13, P=0.001). Participants achieved an average total body weight loss of −2.9% (P=0.008) and controlled attenuation parameter score was reduced by −18.8 dB/m (P=0.021). Mean liver stiffness measurement was unchanged (increase of 0.07 kPA ±3.0, n=17, P=0.411), while an average relative reduction of −20.4% in the Fast score was observed (±71.1, n=17, P=0.011) with 45% (5/11) of participants with a baseline Fast score in the high or indeterminate risk category moving to a lower risk category (FIG. 21).

No serious or device-related adverse events were reported. An average improvement in health-related quality of life of +2.2 Healthy Days per month (P=0.500) and high treatment satisfaction (mean Net Promoter Score of +75) were reported.

Aspects of the present invention are set out in the following clauses:

CLAUSE 1. A computer-implemented method for dynamically treating a subject having a cardiometabolic disorder, the method comprising:
providing, by at least one processor, a digital therapeutic to said subject, the digital therapeutic comprising:
a series of therapy lessons, wherein each therapy lesson addresses one or more of maladaptive beliefs relating to dietary and/or lifestyle behaviors of said subject;
at least one interactive skill-based exercise for each of said therapy lessons to reinforce an improvement with respect to said one or more maladaptive beliefs and/or to initiate a new dietary and/or lifestyle behavior; the method further comprising:
collecting responses from said subject during each said therapy lesson and/or said at least one interactive skill-based exercise, and collecting biometric data from said subject during and/or after each said therapy lesson and/or said at least one interactive skill based exercise;
responsive to the collecting, using at least one treatment processor, providing a progress report characterizing progress by said subject to address said one or more of said maladaptive beliefs, said progress report comprising data relating to the subject's meals and exercise, and data relating to the subject's medication and biometrics; and
responsive to the progress report, recommending one or more goals for said subject to achieve between a current and a next therapy lesson in the series of therapy lessons, the recommending comprising inputting one or more pieces of data in the progress report to one or more algorithms, including one or more machine learning algorithms, so as to provide one or more recommendations for the one or more goals, and wherein the one or more machine learning algorithms are trained using responses and biometric data from a plurality of subjects.

CLAUSE 2. The method according to clause 1, wherein the one or more goals comprise one or more selections from the group consisting of exercise minutes, exercise types, and meals consumed, optionally including one or more lessons and/or skill-building exercises to be completed.

CLAUSE 3. The method according to clause 1, further comprising interacting with said subject so that the subject either accepts the recommended goals, or identifies other goals.

CLAUSE 4. The method according to any preceding clause, wherein the digital therapeutic comprises a treatment plan, the method further comprising, responsive to an extent to which said subject achieves said one or more goals, dynamically adjusting the treatment plan.

CLAUSE 5. The method according to any preceding clause, wherein the cardiometabolic disorder is selected from the group consisting of type 2 diabetes, gestational diabetes, hypertension, obesity, dyslipidemia, hyperlipidemia, hypertriglyceridemia, non-alcoholic fatty liver disease, non-alcoholic steatohepatitis, hypercholesterolemia and familial hypercholesterolemia, heart disease, coronary artery disease, or chronic kidney disease.

CLAUSE 6. The method according to any preceding clause, preferably wherein the one or more recommended goals comprise one or more of diet, exercise and medication.

CLAUSE 7. The method according to any preceding clause, further comprising modifying, for the subject, a subsequent one of said series of therapy lessons and/or said at least one of said interactive skill-based exercises using said at least one processor trained using responses and biometric data from a plurality of subjects, wherein the modifying applies one or more machine learning algorithms.

CLAUSE 8. The method according to any preceding clause, wherein said maladaptive belief is selected from the group comprising or consisting of: ability of the subject to change and/or control behaviors; beliefs of the subject regarding nutrients, optionally including macronutrients, and importance of various food types; or beliefs of the subject regarding experiences in eating and/or exercising.

CLAUSE 9. The method according to any preceding clause, wherein the therapy lesson is specific to the particular condition that the digital therapeutic is treating, or to understanding, addressing, and/or controlling particular human physiological attributes, or to understanding, addressing, and/or controlling particular human physiological responses, or developing certain desirable behaviors.

CLAUSE 10. The method according to any preceding clause, wherein the topic of the therapy lesson relates to one or more of exploring beliefs, Type 2 Diabetes, blood sugar, protein, affordability, exercise, hunger, weight, comfort food, control, loyalty, ability to change, healing, power of beliefs, stress, response to stress, sleep, connection, opportunity, meaning, purpose, strength/resistance exercise, caring for ourselves, empowerment, craving, and/or evolving.

CLAUSE 11. The method according to any preceding clause, wherein the series of therapy lessons comprises two or more, three or more, four or more, five or more, ten or more, fifteen or more, twenty or more, twenty-five or more, or all of said topics.

CLAUSE 12. The method according to any preceding clause, further comprising generating one or more personalized notifications and communicating the one or more personalized notifications to said subject, the one or more personalized notifications selected from the group consisting of reminders, nudges, and rewards.

CLAUSE 13. The method according to clause 12, wherein reminders comprise push notifications to said subject regarding one of said therapy lessons and/or one of said skill-building exercises.

CLAUSE 14. The method according to clause 12 or clause 13, wherein nudges comprise notifications to said subject to direct said subject to a correct next therapy lesson and/or skill-building exercise, or to direct said subject to complete and/or initiate an undone therapy lesson and/or skill-building exercise.

CLAUSE 15. The method according to any of clauses 12 to 14, wherein rewards comprise one or more acknowledgements of successful completion of a therapy lesson and/or skill-building exercise, and/or one or more milestones relating to the subject's meals and/or exercise, and/or relating to the subject's medication and biometrics.

CLAUSE 16. The method according to any preceding clause, wherein the identifying relies at least in part on performance by said subject in reaching previously set goals.

CLAUSE 17. The method according to any preceding clause, wherein said one or more machine learning algorithms is selected from the group comprising or consisting of Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, AdaBoost, or an artificial neural network selected from the group comprising or consisting of convolutional neural networks (CNN), recurrent neural networks (RNN), and recurrent convolutional neural networks (RCNN).

CLAUSE 18. The method according to any preceding clause, wherein one or more of the series of therapy lessons are interactive, and the subject inputs information into the digital therapeutic either voluntarily or as the digital therapeutic provides a prompt.

CLAUSE 19. The method according to clause 18, wherein the information is selected from the group consisting of audio recordings, video recordings, photographs, journal entries, or other written text.

CLAUSE 20. The method according to any preceding clause, wherein the digital therapeutic resides in a smart device selected from the group consisting of a tablet or a smartphone.

CLAUSE 21. The method according to any preceding clause, wherein the collecting comprises the subject entering the subject's biometric data.

CLAUSE 22. The method according to any preceding clause, further comprising providing biometric notifications in response to entry of the subject's biometric data, optionally wherein the biometric notifications indicate danger levels.

CLAUSE 23. The method according to any preceding clause, the method further comprising determining one or more treatment changes and/or behavioral modifications for the subject.

CLAUSE 24. A computer-implemented method for dynamically treating a subject having a cardiometabolic disorder, the method comprising:
providing, by at least one processor, a digital therapeutic to said subject, the digital therapeutic comprising:
a series of therapy lessons, wherein each therapy lesson addresses one or more of maladaptive beliefs relating to dietary and/or lifestyle behaviors of said subject;
at least one interactive skill-based exercise for each of said therapy lessons to reinforce an improvement with respect to said one or more maladaptive beliefs and/or to initiate a new dietary and/or lifestyle behavior;
the method further comprising:
collecting responses from said subject during each said therapy lesson and/or said at least one interactive skill-based exercise, and collecting biometric data from said subject during and/or after each said therapy lesson and/or said at least one interactive skill-based exercise;
responsive to the collecting, using at least one treatment processor, identifying one or more goals for said subject to achieve between a current and a next therapy lesson in the series of therapy lessons, wherein the identifying applies one or more algorithms, including one or more machine learning algorithms, and wherein the one or more machine learning algorithms are trained using responses and biometric data from a plurality of subjects;
responsive to the collecting, using at least one treatment processor, reviewing said subject's progress toward achieving the one or more goals;
responsive to a determination that said subject is not making sufficient progress toward achieving the one or more goals, determining when to send the subject one or more personalized notifications to encourage the subject to provide increased effort to complete one of said therapy lessons and/or perform the at least one interactive skill-based exercise; and
responsive to the determining, sending the subject the one or more personalized notifications to encourage the subject to provide increased effort to complete said one of said therapy lessons and/or perform the at least one interactive skill-based exercise, wherein the determining and the sending employs the one or more algorithms, including the one or more machine learning algorithms, so as to identify a content, timing and/or frequency of the one or more personalized notifications;
wherein the one or more machine learning algorithms are trained using responses and biometric data from a plurality of subjects.

CLAUSE 25. The method according to clause 24, wherein the identifying relies at least in part on performance by said subject in reaching previously-set goals, wherein the identifying applies one or more algorithms, including one or more machine learning algorithms, and wherein the one or more machine learning algorithms is trained using responses and biometric data from a plurality of subjects.

CLAUSE 26. The method according to clause 24 or clause 25, further comprising interacting with said subject so that the subject either accepts the identified goals to be achieved, or identifies other goals to be achieved; and thereby treating said patient when one or more of said goals is achieved.

CLAUSE 27. The method according to any of clauses 24 to 26, wherein the identifying relies at least in part on performance by said subject in reaching previously-set goals, wherein the identifying applies one or more algorithms, including one or more machine learning algorithms, and wherein the one or more machine learning algorithms is trained using responses and biometric data from a plurality of subjects;

CLAUSE 28. The method according to any of clauses 24 to 27, further comprising interacting with said subject so that the subject either accepts the identified goals to be achieved, or identifies other goals to be achieved; and thereby treating said patient when one or more of said goals is achieved.

CLAUSE 29. The method according to any of clauses 24 to 28, wherein the cardiometabolic disorder is selected from the group consisting of type 2 diabetes, gestational diabetes, hypertension, obesity, dyslipidemia, hyperlipidemia, hypertriglyceridemia, non-alcoholic fatty liver disease, non-alcoholic steatohepatitis, hypercholesterolemia and familial hypercholesterolemia, heart disease, coronary artery disease, or chronic kidney disease.

CLAUSE 30. The method according to any of clauses 24 to 29, further comprising dynamically adjusting the goals for the subject between consecutive therapy lessons in said series based on responses and biometric data for a plurality of subjects, wherein the one or more goals comprise one or more of diet, exercise and medication.

CLAUSE 31. The method according to any of clauses 24 to 30, further comprising modifying, for the subject, a subsequent one of said series of therapy lessons and/or said at least one interactive skill-based exercises using said at least one treatment processor, wherein the modifying applies one or more machine learning algorithms trained using responses and biometric data from a plurality of subjects.

CLAUSE 32. The method according to any of clauses 24 to 31, wherein said maladaptive belief is selected from the group comprising or consisting of: ability of the subject to change and/or control behaviors; beliefs of the subject regarding nutrients, optionally including macronutrients, and importance of various food types; or beliefs of the subject regarding experiences in eating and/or exercising.

CLAUSE 33. The method according to any of clauses 24 to 32, wherein the therapy lesson is specific to the particular condition that the digital therapeutic is treating, or to understanding, addressing, and/or controlling particular human physiological attributes, or to understanding, addressing, and/or controlling particular human physiological responses, or developing certain desirable behaviors.

CLAUSE 34. The method according to any of clauses 24 to 33, wherein the topic of the therapy lesson relates to one or more of exploring beliefs, Type 2 Diabetes, blood sugar, protein, affordability, exercise, hunger, weight, comfort food, control, loyalty, ability to change, healing, power of beliefs, stress, response to stress, sleep, connection, opportunity, meaning, purpose, strength/resistance exercise, caring for ourselves, empowerment, craving, and/or evolving.

CLAUSE 35. The method according to any of clauses 24 to 34, wherein the series of therapy lessons comprises two or more, three or more, four or more, five or more, ten or more, fifteen or more, twenty or more, twenty-five or more, or all of said topics.

CLAUSE 36. The method according to any of clauses 24 to 35, wherein the one or more personalized notifications are selected from the group comprising or consisting of reminders, nudges, and rewards.

CLAUSE 37. The method according to clause 36, wherein reminders comprise push notifications to said subject regarding one of said therapy lessons and/or one of said skill-building exercises.

CLAUSE 38. The method according to clause 36, wherein nudges comprise notifications to said subject to direct said subject to a correct next therapy lesson and/or skill-building exercise, or to direct said subject to complete and/or initiate an undone therapy lesson and/or skill-building exercise.

CLAUSE 39. The method according to clause 36, wherein rewards comprise one or more acknowledgements of successful completion of a therapy lesson and/or skill-building exercise, and/or one or more milestones relating to the subject's meals and/or exercise, and/or relating to the subject's medication and biometrics.

CLAUSE 40. The method according to any of clauses 24 to 39, wherein the identifying relies at least in part on performance by said subject in reaching previously set goals.

CLAUSE 41. The method according to any of clauses 24 to 40, wherein said one or more machine learning algorithms is selected from the group comprising or consisting of Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, AdaBoost, or an artificial neural network selected from the group comprising or consisting of convolutional neural networks (CNN), recurrent neural networks (RNN), and recurrent convolutional neural networks (RCNN).

CLAUSE 42. The method according to any of clauses 24 to 41, wherein one or more of the series of therapy lessons are interactive, and the subject inputs information into the digital therapeutic either voluntarily or as the digital therapeutic provides a prompt.

CLAUSE 43. The method according to clause 42, wherein the information is selected from the group consisting of audio recordings, video recordings, photographs, journal entries, or other written text.

CLAUSE 44. The method according to any of clauses 24 to 43, wherein the digital therapeutic resides in a smart device selected from the group consisting of a tablet or a smartphone.

CLAUSE 45. The method according to any of clauses 24 to 44, wherein the collecting comprises the subject entering the subject's biometric data.

CLAUSE 46. The method according to any of clauses 24 to 45, further comprising providing biometric notifications in response to entry of the subject's biometric data, optionally wherein the biometric notifications indicate danger levels.

CLAUSE 47. The method according to any of clauses 24 to 46, further comprising determining one or more treatment changes and/or behavioral modifications for the subject.

CLAUSE 48. A computer-implemented method for dynamically adjusting a treatment plan for a subject having a cardiometabolic disorder, the method comprising:
providing, by at least one processor, a digital therapeutic to said subject, the digital therapeutic comprising a treatment plan comprising:
a series of therapy lessons, wherein each therapy lesson addresses one or more of maladaptive beliefs relating to dietary and/or lifestyle behaviors of said subject;
at least one interactive skill-based exercise for each of said therapy lessons to reinforce an improvement with respect to said one or more maladaptive beliefs and/or to initiate a new dietary and/or lifestyle behavior;
the method further comprising:
collecting responses from said subject during each said therapy lesson and/or said at least one interactive skill-based exercise, and collecting biometric data from said subject during and/or after each said therapy lesson and/or said at least one interactive skill-based exercise;
responsive to the collecting, using at least one treatment processor, identifying one or more goals for said subject to achieve between a current and a next therapy lesson in the series of therapy lessons, wherein the identifying applies one or more algorithms, including one or more machine learning algorithms, and wherein the one or more machine learning algorithms are trained using responses and biometric data from a plurality of subjects;
responsive to the collecting, using at least one treatment processor, reviewing said subject's progress toward achieving the one or more goals; and
responsive to a determination of said subject's progress toward achieving the one or more goals, performing one of the following to dynamically adjust the treatment plan:
repeating the current therapy lesson;
repeating an earlier one of the series of therapy lessons; or
skipping one or more of the series of therapy lessons following the current therapy lesson;
wherein the determination employs the one or more algorithms, including the one or more machine learning algorithms.

CLAUSE 49. The method according to clause 48, wherein the determination of said subject's progress includes an assessment of passage of time since said subject completed a particular therapy lesson and/or skill-building exercise.

CLAUSE 50. The method according to clause 48, wherein the determination of said subject's progress includes an assessment of whether said subject completed a particular therapy lesson and/or skill-building exercise.

CLAUSE 51. The method according to clause 48, wherein the determination of said subject's progress includes an assessment of how well said subject completed a particular therapy lesson and/or skill-building exercise.

CLAUSE 52. The method according to any of clauses 48 to 51, wherein the identifying relies at least in part on performance by said subject in reaching previously-set goals, wherein the identifying applies one or more algorithms, including one or more machine learning algorithms, and wherein the one or more machine learning algorithms is trained using responses and biometric data from a plurality of subjects.

CLAUSE 53. The method according to any of clauses 48 to 52, further comprising interacting with said subject so that the subject either accepts the identified goals to be achieved, or identifies other goals to be achieved; and thereby treating said patient when one or more of said goals is achieved.

CLAUSE 54. The method according to any of clauses 48 to 53, the method further comprising dynamically adjusting the goals for the subject between consecutive therapy lessons in said series using said at least one treatment processor, wherein the dynamic adjustment applies one or more machine learning algorithms trained using responses and biometric data from a plurality of subjects, preferably wherein the one or more goals comprise one or more of diet, exercise and medication.

CLAUSE 55. The method according to any of clauses 48 to 54, the method further comprising modifying, for the subject, a subsequent one of said series of therapy lessons and/or said at least one interactive skill-based exercises using said at least one treatment processor, wherein the modifying applies one or more machine learning algorithms trained using responses and biometric data from a plurality of subjects.

CLAUSE 56. The method according to any of clauses 48 to 55, wherein said maladaptive belief is selected from the group comprising or consisting of: ability of the subject to change and/or control behaviors; beliefs of the subject regarding nutrients, optionally including macronutrients, and importance of various food types; or beliefs of the subject regarding experiences in eating and/or exercising.

CLAUSE 57. The method according to any of clauses 48 to 56, wherein the therapy lesson is specific to the particular condition that the digital therapeutic is treating, or to understanding, addressing, and/or controlling particular human physiological attributes, or to understanding, addressing, and/or controlling particular human physiological responses, or developing certain desirable behaviors.

CLAUSE 58. The method according to any of clauses 48 to 57, wherein the topic of the therapy lesson is selected from the group comprising or consisting of: exploring beliefs, Type 2Diabetes, blood sugar, protein, affordability, exercise, hunger, weight, comfort food, control, loyalty, ability to change, healing, power of beliefs, stress, response to stress, sleep, connection, opportunity, meaning, purpose, strength/resistance exercise, caring for ourselves, empowerment, craving, and/or evolving.

CLAUSE 59. The method according to any of clauses 48 to 58, wherein the series of therapy lessons comprises two or more, three or more, four or more, five or more, ten or more, fifteen or more, twenty or more, twenty-five or more, or all of said topics.

CLAUSE 60. The method according to any of clauses 48 to 59, the method further comprising generating one or more personalized notifications and communicating the one or more personalized notifications to said subject, the one or more personalized notifications selected from the group consisting of reminders, nudges, and rewards.

CLAUSE 61. The method according to any of clauses 48 to 60, wherein the identifying relies at least in part on performance by said subject in reaching previously set goals.

CLAUSE 62. The method according to any of clauses 48 to 61, wherein said one or more machine learning algorithms is selected from the group comprising or consisting of Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, AdaBoost, or an artificial neural network selected from the group comprising or consisting of convolutional neural networks (CNN), recurrent neural networks (RNN), and recurrent convolutional neural networks (RCNN).

CLAUSE 63. The method according to any of clauses 48 to 62, wherein one or more of the series of therapy lessons are interactive, and the subject inputs information into the digital therapeutic either voluntarily or as the digital therapeutic provides a prompt.

CLAUSE 64. The method according to clause 63, wherein the information is selected from the group consisting of audio recordings, video recordings, photographs, journal entries, or other written text.

CLAUSE 65. The method according to any of clauses 48 to 64, wherein the digital therapeutic resides in a smart device selected from the group consisting of a tablet or a smartphone.

CLAUSE 66. The method according to any of clauses 48 to 65, wherein the collecting comprises the subject entering the subject's biometric data.

CLAUSE 67. The method according to any of clauses 48 to 66, the method further comprising providing biometric notifications in response to entry of the subject's biometric data, optionally wherein the biometric notifications indicate danger levels.

CLAUSE 68. The method according to any of clauses 48 to 67, the method further comprising determining one or more treatment changes and/or behavioral modifications for the subject.

CLAUSE 69. A computer-implemented method for dynamically treating a subject having a cardiometabolic disorder, the method comprising:
providing, by at least one processor, a digital therapeutic to said subject, the digital therapeutic comprising:
a series of therapy lessons, wherein each therapy lesson addresses one or more of maladaptive beliefs relating to dietary and/or lifestyle behaviors of said subject;
at least one interactive skill-based exercise for each of said therapy lessons to reinforce an improvement with respect to said one or more maladaptive beliefs and/or to initiate a new dietary and/or lifestyle behavior;
the method further comprising:
collecting responses from said subject during each said therapy lesson and/or said at least one interactive skill-based exercise, and collecting biometric data from said subject during and/or after each said therapy lesson and/or said at least one interactive skill-based exercise;
responsive to the collecting, using at least one treatment processor, identifying one or more
goals for said subject to achieve between a current and a next therapy lesson in the series of therapy lessons, wherein the identifying applies one or more algorithms, including one or more machine learning algorithms, and wherein the one or more machine learning algorithms are trained using responses and biometric data from a plurality of subjects;
responsive to the collecting, using at least one treatment processor, reviewing said subject's progress toward achieving the one or more goals; and
responsive to a determination of said subject's progress toward achieving the one or more goals, performing one of the following to dynamically treat the subject:
repeating the current therapy lesson;
repeating an earlier one of the series of therapy lessons; or
skipping one or more of the series of therapy lessons following the current therapy lesson;
wherein the determination employs the one or more algorithms, including the one or more machine learning algorithms.

CLAUSE 70. The method according to clause 69, wherein the determination of said subject's progress includes an assessment of passage of time since said subject completed a particular therapy lesson and/or skill-building exercise.

CLAUSE 71. The method according to clause 69, wherein the determination of said subject's progress includes an assessment of whether said subject completed a particular therapy lesson and/or skill-building exercise.

CLAUSE 72. The method according to clause 69, wherein the determination of said subject's progress includes an assessment of how well said subject completed a particular therapy lesson and/or skill-building exercise.

CLAUSE 73. The method according to any of clauses 69 to 72, wherein the cardiometabolic disorder is selected from the group consisting of type 2 diabetes, gestational diabetes, hypertension, obesity, dyslipidemia, hyperlipidemia, hypertriglyceridemia, non-alcoholic fatty liver disease, non-alcoholic steatohepatitis, hypercholesterolemia and familial hypercholesterolemia, heart disease, coronary artery disease, or chronic kidney disease.

CLAUSE 74. The method according to any of clauses 69 to 73, further comprising dynamically adjusting the goals for the subject between consecutive therapy lessons in said series using said at least one treatment processor, wherein the dynamic adjustment applies one or more machine learning algorithms trained using responses and biometric data from a plurality of subjects, preferably wherein the one or more goals comprise one or more of diet, exercise and medication.

CLAUSE 75. The method according to any of clauses 69 to 74, the method further comprising modifying, for the subject, a subsequent one of said series of therapy lessons and/or said at least one interactive skill-based exercises using said at least one processor trained using responses and biometric data from a plurality of subjects, wherein the modifying applies one or more machine learning algorithms.

CLAUSE 76. The method according to any of clauses 69 to 75, wherein said maladaptive belief is selected from the group comprising or consisting of: ability of the subject to change and/or control behaviors; beliefs of the subject regarding nutrients, optionally including macronutrients, and importance of various food types; or beliefs of the subject regarding experiences in eating and/or exercising.

CLAUSE 77. The method according to any of clauses 69 to 76, wherein the therapy lesson is specific to the particular condition that the digital therapeutic is treating, or to understanding, addressing, and/or controlling particular human physiological attributes, or to understanding, addressing, and/or controlling particular human physiological responses, or developing certain desirable behaviors.

CLAUSE 78. The method according to any of clauses 69 to 77, wherein the topic of the therapy lesson relates to one or more of exploring beliefs, Type 2 Diabetes, blood sugar, protein, affordability, exercise, hunger, weight, comfort food, control, loyalty, ability to change, healing, power of beliefs, stress, response to stress, sleep, connection, opportunity, meaning, purpose, strength/resistance exercise, caring for ourselves, empowerment, craving, and/or evolving.

CLAUSE 79. The method according to any of clauses 69 to 78, wherein the series of therapy lessons comprises two or more, three or more, four or more, five or more, ten or more, fifteen or more, twenty or more, twenty-five or more, or all of said topics

CLAUSE 80. The method according to any of clauses 69 to 79, the method further comprising generating one or more personalized notifications and communicating the one or more personalized notifications to said subject, the one or more personalized notifications selected from the group consisting of reminders, nudges, and rewards.

CLAUSE 81. The method according to any of clauses 69 to 80, wherein the identifying relies at least in part on performance by said subject in reaching previously set goals.

CLAUSE 82. The method according to any of clauses 69 to 81, wherein said one or more machine learning algorithms is selected from the group comprising or consisting of Linear Regression, Logistic Regression, Linear Discriminant Analysis, Classification and Regression Trees, Decision Trees, Clustering, Inductive Logic Processing, Stochastic Modeling, Statistical Modeling, Case-Based Reasoning, Bayes and Naive Bayes, K-Nearest Neighbors (KNN), Learning Vector Quantization (LVQ), Support Vector Machines (SVM), Random Forest, Boosting, AdaBoost, or an artificial neural network selected from the group comprising or consisting of convolutional neural networks (CNN), recurrent neural networks (RNN), and recurrent convolutional neural networks (RCNN).

CLAUSE 83. The method according to any of clauses 69 to 82, wherein one or more of the series of therapy lessons are interactive, and the subject inputs information into the digital therapeutic either voluntarily or as the digital therapeutic provides a prompt.

CLAUSE 84. The method according to any of clauses 69 to 83, wherein the information is selected from the group consisting of audio recordings, video recordings, photographs, journal entries, or other written text.

CLAUSE 85. The method according to any of clauses 69 to 84, wherein the digital therapeutic resides in a smart device selected from the group consisting of a tablet or a smartphone.

CLAUSE 86. The method according to any of clauses 69 to 85, wherein the collecting comprises the subject entering the subject's biometric data.

CLAUSE 87. The method according to any of clauses 69 to 86, the method further comprising providing biometric notifications in response to entry of the subject's biometric data, optionally wherein the biometric notifications indicate danger levels.

CLAUSE 88. The method according to any of clauses 69 to 87, the method further comprising determining one or more treatment changes and/or behavioral modifications for the subject.

While aspects of the present invention have been described in detail above with reference to one or more embodiments, variations within the scope and spirit of the invention will be apparent to ordinarily skilled artisans. Accordingly, the invention should be considered as limited only by reference to the following claims.

What is claimed is:

1. A method, comprising:
providing, by one or more processors, a digital therapeutic application to a user comprising one or more lessons or activities;
collecting, by the one or more processors, at least one response or biometric data from the user corresponding to the one or more lessons or activities; and
generating, by the one or more processors using a machine-learning (ML) model, (a) one or more goals for the user to achieve based on the at least one response or biometric data or (b) a progress overview based on a progress by the user to achieve one or more goals.

2. The method of claim 1, further comprising:
responsive to the progress overview, recommending, by the one or more processors using the ML model, one or more new goals for the user to achieve between a current lesson or activity and a next lesson or activity in a series of lessons or activities, wherein a portion of the progress overview is used as input to the ML model.

3. The method of claim 2, further comprising:
providing, by the one or more processors, the one or more new goals for the user to achieve between the current lesson or activity and the next lesson.

4. The method of claim 1, wherein the progress overview comprises data corresponding to at least one meal or exercise of the user, and the data further comprises one or more medications or one or more biometrics of the user.

5. The method of claim 1, wherein the progress of the user is based at least on performance by the user in reaching previously-set goals.

6. The method of claim 5, wherein the progress of the user corresponds to at least one of a passage of time since the user completed a lesson or activity, whether the user completed a lesson or activity, or how the user completed a particular lesson or activity.

7. The method of claim 1, further comprising:
receiving or identifying, by the one or more processors, an indication of performance one or more actions to cause an update in a treatment plan.

8. The method of claim 7, wherein the one or more actions comprise (i) repeating a current lesson or activity, (ii) repeating an earlier lesson or activity, or (iii) skipping at least one of the one or more lessons or activities.

9. The method of claim 7, wherein the treatment plan comprises a series of lessons or activities to address one or more maladaptive beliefs corresponding to dietary or lifestyle behaviors of the user, wherein updating the treatment plan is based at least on performance by the user in reaching previously-set goals.

10. The method of claim 1, wherein the progress overview characterizes progress by the user to address one or more maladaptive beliefs of the user.

11. The method of claim 1, further comprising:
interfacing, by the one or more processors, with the user to cause at least one of (i) an acceptance of the one or more goals to be achieved or (ii) an identification of one or more new goals to be achieved.

12. The method of claim 1, wherein the one or more lessons or activities correspond to addressing a cardiometabolic disorder of the user, the cardiometabolic disorder comprises at least one of type 2 diabetes, gestational diabetes, hypertension, obesity, dyslipidemia, hyperlipidemia, hypertriglyceridemia, non-alcoholic fatty liver disease, non-alcoholic steatohepatitis, hypercholesterolemia and familial hypercholesterolemia, heart disease, coronary artery disease, or chronic kidney disease.

13. The method of claim 1, wherein the one or more lessons or activities correspond to at least one of exploring beliefs, type 2 diabetes, blood sugar, protein, affordability, activity, hunger, weight, comfort food, control, loyalty, ability to change, healing, power of beliefs, stress, response to stress, sleep, connection, opportunity, meaning, purpose, strength or resistance activities, caring for oneself, empowerment, craving, or evolving.

14. The method of claim 1, further comprising:
updating, by the one or more processors using the ML model, one or more subsequent lessons or activities based on the at least one response or biometric data.

15. The method of claim 1, further comprising:
transmitting, by the one or more processors, one or more personalized notifications corresponding to at least one of a reminder, nudge, or reward.

16. The method of claim 15, wherein the reminder corresponds to a push notification to the user for the one or more lessons or activities, and wherein the nudge corresponds to a notification to the user encouraging the user to a next lesson or activity, or to direct the user to complete or initiate an uncompleted lesson or activity, and wherein the reward corresponds to an acknowledgement of completion of a lesson or activity, or one or more milestones corresponding to at least one of a medication, or a biometric of the user.

17. The method of claim 1, wherein the one or more lessons or activities correspond to one or more interactive lessons or activities, and wherein the at least one response or biometric data is collected via voluntary user input on the digital therapeutic application or in response to a prompt, and wherein the at least one response comprises at least one of an audio recording, a video recording, a photograph, or a journal entry.

18. The method of claim 1, further comprising:
administering an effective amount of any one or more of: metformin, sulfonylureas, sglt2 inhibitors, glp-1 analogues, insulin, dpp-4 inhibitors, thiazolidinediones, meglitinides, glipizide, glimepiride, glyburide, repaglinide, nateglinide, pioglitazone, rosiglitazone, sitagliptin, saxagliptin, linagliptin, alogliptin, canagliflozin, dapagliflozin, empagliflozin, liraglutide, semaglutide, tirzepatide, acarbose, insulin glulisine, insulin lispro, insulin aspart, insulin glargine, insulin detemir, insulin isophane, colesevelam, bromocriptine, or pramlintide.

19. The method of claim 1, wherein the user is taking a medication for type 2 diabetes, the medication selected from any one of: metformin, sulfonylureas, sglt2 inhibitors, glp-1 analogues, insulin, dpp-4 inhibitors, thiazolidinediones, meglitinides, glipizide, glimepiride, glyburide, repaglinide, nateglinide, pioglitazone, rosiglitazone, sitagliptin, saxagliptin, linagliptin, alogliptin, canagliflozin, dapagliflozin, empagliflozin, liraglutide, semaglutide, tirzepatide, acarbose, insulin glulisine, insulin lispro, insulin aspart, insulin glargine, insulin detemir, insulin isophane, colesevelam, bromocriptine, or pramlintide.

20. A system, comprising:
one or more processors coupled with memory, configured to:
present, via a device, a digital therapeutic comprising at least one lesson, the at least one lesson corresponding to at least one interactive skill-based activity;
transmit instructions to the device to instruct a user to complete the at least one lesson over a treatment interval; and
responsive to receiving lesson completion data, transmit a prompt directing the user to adjust at least one of a dietary intake or a physical-activity behavior;
wherein adjusting the dietary intake or the physical-activity behavior corresponds to a reduction in at least one biomarker level of the user, the at least one biomarker level corresponding to a characteristic of type 2 diabetes, by at least a percent relative to a baseline level of the at least one biomarker.

21. The system of claim 20, wherein presenting the digital therapeutic further comprises presenting one or more goals corresponding to one or more selections from the group consisting of exercise, exercise minutes, exercise types, diet, meals consumed, and medication.

22. The system of claim 21, wherein the one or more processors are further configured to:
transmit a prompt directing the user to either accept the one or more goals or identify other goals.

23. The system of claim 20, wherein the digital therapeutic comprises a treatment plan, wherein the one or more processors are further configured to:
responsive to an extent to which the user achieves one or more goals, dynamically adjust the treatment plan.

24. The system of claim 20, wherein the at least one lesson is specific to treating type-2 diabetes such that the digital therapeutic is understanding, addressing, or controlling particular human physiological attributes, physiological responses, or developing certain desirable behaviors.

25. The system of claim 20, wherein the at least one lesson or at least one activity relates to one or more of exploring beliefs, Type 2 Diabetes, blood sugar, protein, affordability, exercise, hunger, weight, comfort food, control, loyalty, ability to change, healing, power of beliefs, stress, response to stress, sleep, connection, opportunity, meaning, purpose, strength/resistance exercise, caring for ourselves, empowerment, craving, or evolving.

26. The system of claim 20, wherein the one or more processors are further configured to:
provide a progress overview generated by a machine-learning (ML) model to the user device.

27. The system of claim 26, wherein the one or more processors are further configured to:
responsive to receiving lesson or activity completion data, generate or recommend, by the ML model, one or more new lessons of the digital therapeutic.

28. The system of claim 20, wherein the at least one biomarker comprises biometric data, blood sugar levels, blood pressure, heartbeat, weight, physiological responses, alanine transaminase, or liver fat.

29. The system of claim 20, wherein the user is taking a medication for type 2 diabetes, the medication selected from any one of: metformin, sulfonylureas, sglt2 inhibitors, glp-1 analogues, insulin, dpp-4 inhibitors, thiazolidinediones, meglitinides, glipizide, glimepiride, glyburide, repaglinide, nateglinide, pioglitazone, rosiglitazone, sitagliptin, saxagliptin, linagliptin, alogliptin, canagliflozin, dapagliflozin, empagliflozin, liraglutide, semaglutide, tirzepatide, acarbose, insulin glulisine, insulin lispro, insulin aspart, insulin glargine, insulin detemir, insulin isophane, colesevelam, bromocriptine, or pramlintide.

30. A non-transitory computer readable medium (CRM) comprising one or more instructions stored thereon and executable by one or more processors to:
provide a digital therapeutic application to a user comprising one or more lessons or activities;
collect at least one response or biometric data from the user corresponding to the one or more lessons or activities; and
generate, using a machine-learning (ML) model, (a) one or more goals for the user to achieve based on the at least one response or biometric data or (b) a progress overview based on a progress by the user to achieve one or more goals.
